(19) United States
(12) Patent Application Publication
(10) Pub. No.: US 2025/0366809 A1
(43) Pub. Date: Dec. 4, 2025

(54) X-RAY CT APPARATUS, INFORMATION PROCESSING APPARATUS, INFORMATION PROCESSING METHOD, AND RECORDING MEDIUM

(71) Applicant: CANON MEDICAL SYSTEMS CORPORATION, Otawara-shi (JP)

(72) Inventors: Gakuto AOYAMA, Kawasaki (JP); Yoshinori HIRANO, Nasushiobara (JP)

(73) Assignee: CANON MEDICAL SYSTEMS CORPORATION, Otawara-shi (JP)

(21) Appl. No.: 19/214,415

(22) Filed: May 21, 2025

(30) Foreign Application Priority Data
May 30, 2024 (JP) 2024-088077
Mar. 7, 2025 (JP) 2025-036385

(51) Int. Cl. A61B 6/00; A61B 6/03; A61B 6/46; G06F 3/04817; G06F 16/11; G16H 10/60; G16H 30/20; G16H 40/20

(52) U.S. Cl. CPC A61B 6/5211; A61B 6/03; A61B 6/465; G06F 3/04817; G06F 16/125; G16H 10/60; G16H 30/20; G16H 40/20

(57) ABSTRACT

An X-ray CT apparatus according to an embodiment includes an X-ray tube, an X-ray detector, a memory, and processing circuitry connected to the memory. The X-ray tube irradiates a subject with X-rays. The X-ray detector detects X-rays emitted from the X-ray tube. The processing circuitry generates a prompt. The processing circuitry inputs the prompt to a generative model and acquires generative information from the generative model. The memory stores, as storage target data, the prompt out of the generative information and the prompt corresponding to the generative information. An embodiment discloses an information processing apparatus including the memory and the processing circuitry connected to the memory, an information processing method, and a recording medium.

### CROSS-REFERENCE TO RELATED APPLICATIONS

This application is based upon and claims the benefit of priority from Japanese Patent Application No. 2024-088077, filed on May 30, 2024; and Japanese Patent Application No. 2025-036385, filed on Mar. 7, 2025; the entire contents of all of which are incorporated herein by reference.

### FIELD

Embodiments disclosed herein relate generally to an X-ray CT apparatus, an information processing apparatus, an information processing method, and a recording medium.

### BACKGROUND

In recent years, the utilization of generative information generated by generative artificial intelligence (AI) has been developed. Storing templates for prompts has also been done (e.g., Japanese Patent. No. 7416390B1). Such technology allows for easy generation of image data and the like by inputting prompts to the generative AI. In particular, in the medical field, it is common for various information referred to by physicians for diagnosis to be saved for a certain period of time. However, if all the generated generative information is to be saved for a long term, the amount of data saved increases.

### BRIEF DESCRIPTION OF THE DRAWINGS

FIG. 1 is a diagram illustrating an exemplary overall configuration of an information processing system according to a first embodiment;

FIG. 2 is a diagram illustrating an exemplary input to and an exemplary output from a generative model according to the first embodiment;

FIG. 3 is a diagram illustrating an exemplary display screen according to the first embodiment;

FIG. 4 is a flowchart illustrating an exemplary procedure of a process related to generation and saving of generative information according to the first embodiment;

FIG. 5 is a flowchart illustrating an exemplary procedure of a process of deleting storage target data based on a deletion condition according to the first embodiment;

FIG. 6 is a diagram illustrating an exemplary display screen according to a second embodiment;

FIG. 7 is a diagram illustrating an exemplary state in which a store button is pressed by a user in FIG. 6;

FIG. 8 is a diagram illustrating an exemplary display screen according to a third embodiment;

FIG. 9 is a diagram illustrating an exemplary overall configuration of the information processing system according to a fourth embodiment;

FIG. 10 is a diagram illustrating an exemplary relationship between a version of a generative model and generative information 410 according to a sixth embodiment;

FIG. 11 is a diagram illustrating an exemplary relationship between versions of generative models and generative information when a prompt is re-input according to a seventh embodiment;

FIG. 12 is a diagram illustrating an example of determination of storage target data based on similarity of multiple pieces of generative information according to a ninth embodiment;

FIG. 13 is a diagram illustrating another example of determination of storage target data based on similarity of multiple pieces of generative information according to the ninth embodiment;

FIG. 14 is a diagram illustrating an example of storage target data according to a tenth embodiment;

FIG. 15 is a diagram illustrating an exemplary overall configuration of the information processing system according to an eleventh embodiment; and

FIG. 16 is a diagram illustrating an exemplary hardware configuration of an X-ray CT apparatus according to the eleventh embodiment.

### DETAILED DESCRIPTION

Embodiments of an X-ray CT apparatus, an information processing apparatus, an information processing method, and a recording medium will be described in detail below with reference to the drawings.

An X-ray CT apparatus according to an embodiment includes an X-ray tube, an X-ray detector, at least one memory, and at least one piece of processing circuitry connected to the memory. The X-ray tube is configured to irradiate a subject with X-rays. The X-ray detector is configured to detect X-rays emitted from the X-ray tube. The processing circuitry is configured to generate a prompt. The processing circuitry is configured to input the prompt to a generative model and acquire generative information from the generative model. The memory is configured to store, as storage target data, the prompt out of the generative information and the prompt corresponding to the generative information.

An information processing apparatus according to an embodiment includes at least one memory and at least one piece of processing circuitry connected to the memory. The processing circuitry is configured to generate a prompt. The processing circuitry is configured to input the prompt to a generative model and acquire generative information from the generative model. The memory is configured to store, as storage target data, the prompt out of the generative information and the prompt corresponding to the generative information.

### First Embodiment

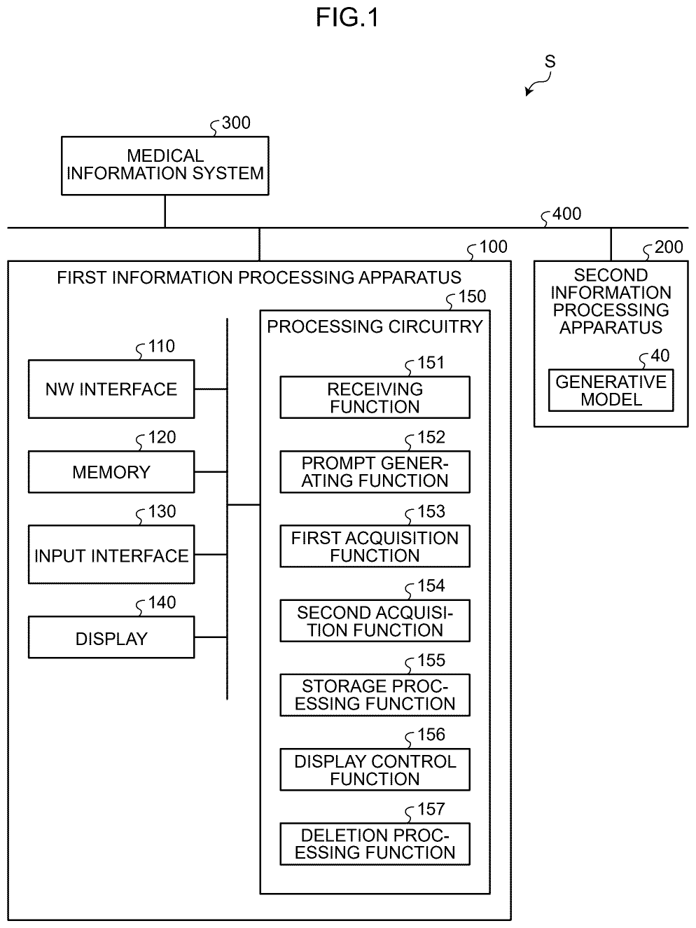

FIG. 1 is a diagram illustrating an exemplary overall configuration of an information processing system S according to a first embodiment. As illustrated in FIG. 1, the information processing system S includes, as an example, a first information processing apparatus 100, a second information processing apparatus 200, and a medical information system 300. The information processing system S is installed in a medical institution, such as a hospital. The first information processing apparatus 100, the second information processing apparatus 200, and the medical information system 300 are communicatively connected via a network 400, such as a hospital local area network (LAN).

The medical information system 300 is a system that saves and manages medical information and includes, for example, one or more servers or personal computers (PCs). The medical information saved in the medical information system 300 is, for example, patient's electronic medical records, clinical information, results of medical interviews, test data, medical image data, and diagnostic results. The medical information system 300 may have, for example, a function such as an electronic medical record system, a hospital information system (HIS), a laboratory information system (LIS), a radiology information system (RIS), or a medical-use image archive device. The medical-use image archive device is, for example, a server device for a picture archiving and communication system (PACS) and in which medical-use image (medical image) data is archived in a format compliant with digital imaging and communications in medicine (DICOM). The medical information system 300 may include more than one of the systems, apparatuses, and the like listed above.

The first information processing apparatus 100 and the second information processing apparatus 200 are, for example, computers such as servers or PCs.

The second information processing apparatus 200 includes a trained model of generative artificial intelligence (AI) (hereinafter referred to as generative model 40). The second information processing apparatus 200 includes, for example, a memory and a processor. The generative model 40 is, for example, stored in the memory in the second information processing apparatus 200 and operated by the processor. The configuration of the second information processing apparatus 200 is not particularly limited, and any known configuration can be employed.

When receiving an input of a prompt, the generative model 40 generates information corresponding to the prompt and outputs the generated information. The generative model 40 is, for example, a multimodal model (MMM) or a large language model (LLM) that can handle multiple types of data such as image data, and is an integrated AI model that can process multiple types of modalities (data types) such as text, images, voice, and numeric values at once.

The information generated by the generative model 40 is referred to as generative information. The generative information includes, for example, image data. The generative information may include text as well as image data. Depending on the content of the prompt, the generative information does not necessarily include image data. In the present embodiment, the information processing system S is used in the medical field, so generative information 410 includes, for example, information about a patient. The information about a patient is, for example, medical information. The information about a patient may be information about the patient's gender, age, and body size. The generative information is, for example, medical image data. Hereafter, the image data may be simply referred to as “image”. The medical image is also referred to as medical-use image.

The prompt input to the generative model 40 is typically text data. The prompt may include image data. The prompt includes an instruction specifying the content of generative information to be generated by the generative model 40. Since the information processing system S in the present embodiment is used in, for example, the medical field, the prompt includes medical information. External information not mentioned in the body of the prompt may be referred to in the prompt. For example, the generative model 40 may retrieve external information and use the retrieved external information to generate generative information, like retrieval-augmented generation (RAG). The external information to be retrieved includes, for example, various medical information saved in the medical information system 300.

The first information processing apparatus 100 is operated by a user and provides the user with an output result from the generative model 40 stored in the second information processing apparatus 200. The user of the first information processing apparatus 100 is, for example, a physician or a medical professional, who uses the generative model 40 via the first information processing apparatus 100 to obtain information to be referred to for diagnosis of a patient. The first information processing apparatus 100 is an example of the information processing apparatus in the present disclosure.

The first information processing apparatus 100 includes a network (NW) interface 110, a memory 120, an input interface 130, a display 140, and processing circuitry 150.

The NW interface 110 is connected to the processing circuitry 150 and controls transmission and communication of various data between the first information processing apparatus 100, and the second information processing apparatus 200, and the medical information system 300. The NW interface 110 is implemented by a network card, a network adapter, a network interface controller (NIC), or the like.

The memory 120 stores various information to be used by the processing circuitry 150 in advance. The memory 120 stores various computer programs. The memory 120 is, for example, a non-volatile storage device, such as a hard disk drive (HDD), a solid state drive (SSD), or an integrated circuit storage device, that stores various information. The memory 120 may be a drive device, other than an HDD or an SSD, that reads and writes various information from/into a portable recording medium such as a compact disc (CD), a digital versatile disc (DVD), or a flash memory, or a semiconductor memory device such as a random access memory (RAM). The memory 120 is an example of a memory unit.

The memory 120 stores the prompt out of the generative information generated by the generative model 40 and the prompt corresponding to the generative information, as storage target data. The prompt corresponding to the generative information refers to a prompt that is input for generating generative information. In the present embodiment, the memory 120 stores at least a prompt as storage target data. When generative information is specified as a storage target by a user operation, the memory 120 may store the generative information as storage target data.

In the present embodiment, the storage target data is not data that is temporarily saved during processing but data that is saved for a long term in the memory 120. In the present embodiment, the expression “store as storage target data” means storing on the assumption of being saved for a long term. The simple expression “save” encompasses both saving temporarily and saving for a long term. To save temporarily during work until the physician's diagnosis is confirmed is different from to store as storage target data. The storage target data is also referred to as data to be saved for a long term.

A saving period in saving for a long term is, for example, 5-years when the generative information is information related to medical care and used for patient diagnosis and linked to an electronic medical record. However, this period is an example and the saving period is not limited to this. The storage target data may be kept saved for more than 5-years.

Unlike cache data or the like, which may be automatically erased or overwritten, the storage target data stored in the memory 120 is not deleted except when a predetermined deletion condition is met or when a deletion operation is executed by the user. Details of saving and deleting a prompt and generative information in the memory 120 will be described later.

The input interface 130 is implemented by a mouse, a keyboard, a pen tablet that combines a touch pen and a tablet to receive an operation by the user, a trackball, a switch button, a touchpad that allows for an input operation by touching an operation surface, a touchscreen that integrates a display screen and a touchpad, a non-contact input circuit using an optical sensor, an audio input circuit, or the like. The input interface 130 may include multiple devices that receive an operation by the user. The input interface 130 is connected to the processing circuitry 150 to convert an input operation received from the user into an electrical signal and output the electrical signal to the processing circuitry 150. As used herein, the input interface is not limited to those with physical operating components, such as a mouse and a keyboard. For example, an electrical signal processing circuit that receives an electrical signal corresponding to an input operation from an external input device installed separately from the apparatus and outputs the electrical signal to the processing circuitry 150 is also an example of the input interface.

The display 140 displays various information under the control of the processing circuitry 150. For example, the display 140 outputs a screen containing generative information generated by the generative model 40, a graphical user interface (GUI) for receiving various operations from the user, and the like. The display 140 is specifically a liquid crystal display, a cathode ray tube (CRT) display, or the like. The input interface 130 and the display 140 may be integrated. For example, the input interface 130 and the display 140 may be implemented by a touch panel.

The processing circuitry 150 is a processor that reads computer programs from the memory 120 and executes the computer programs to implement a function corresponding to each of the computer programs. The processing circuitry 150 in the present embodiment includes an receiving function 151, a prompt generating function 152, a first acquisition function 153, a second acquisition function 154, a storage processing function 155, a display control function 156, and a deletion processing function 157. The receiving function 151 is an example of an receiving unit. The prompt generating function 152 is an example of a prompt generating unit. The first acquisition function 153 is an example of a first acquisition unit or an acquisition unit. The second acquisition function 154 is an example of a second acquisition unit or an acquisition unit. The storage processing function 155 is an example of a storage processing unit. The display control function 156 is an example of a display control unit. The deletion processing function 157 is an example of a deletion processing unit.

Here, each of the processing functions including the receiving function 151, the prompt generating function 152, the first acquisition function 153, the second acquisition function 154, the storage processing function 155, the display control function 156, and the deletion processing function 157, which are components of the processing circuitry 150, is stored in the memory 120 in the form of a computer program executable by a computer. The processing circuitry 150 is a processor. For example, the processing circuitry 150 reads computer programs from the memory 120 and executes the computer programs to implement a function corresponding to each of the computer programs. In other words, the processing circuitry 150 in a state in which the computer programs are read out has each function depicted in the processing circuitry 150 in FIG. 1. In FIG. 1, a single processor implements the processing functions performed by the receiving function 151, the prompt generating function 152, the first acquisition function 153, the second acquisition function 154, the storage processing function 155, the display control function 156, and the deletion processing function 157. Alternatively, plural independent processors may be combined to construct the processing circuitry 150 such that each processor executes a computer program to implement a corresponding function. In FIG. 1, the single memory 120 stores the computer program corresponding to each processing function. Alternatively, plural memories may be distributed and allocated, and the processing circuitry 150 may be configured to read a corresponding computer program from each individual memory.

In the above explanation, an example in which a “processor” reads and executes a computer program corresponding to each function from a memory. However, the embodiments are not limited to this. The term “processor” means circuitry such as a central processing unit (CPU), a graphics processing unit (GPU), an application specific integrated circuit (ASIC), or a programmable logic device (e.g., simple programmable logic device (SPLD), complex programmable logic device (CPLD), and a field programmable gate array (FPGA)). When, for example, the processor is a CPU, the processor reads and executes a computer program saved in a memory to implement a function. On the other hand, when the processor is an ASIC, instead of saving a computer program in the memory 120, the function is directly built in circuitry of the processor as logic circuitry. Each processor in the present embodiment is not limited to a single piece of circuitry configured for each processor, but multiple pieces of independent circuitry may be combined as one processor to implement the objective function. Moreover, a plurality of components in FIG. 1 may be integrated into a single processor to implement the objective function.

The receiving function 151 receives various user operations via the input interface 130. For example, the receiving function 151 receives an operation of inputting a prompt to be input to the generative model 40, an editing operation, and an operation to specify storage target data by the user. For example, the receiving function 151 receives a user operation to specify the generative information 410 as storage target data.

The prompt generating function 152 generates a prompt to be input to the generative model 40. For example, the prompt generating function 152 generates a prompt based on the user operation received by the receiving function 151. The prompt generating function 152 may automatically generate part or the whole of the prompt in accordance with a predetermined rule. The prompt generating function 152 may have a function to check the description of a prompt 50 input by the user.

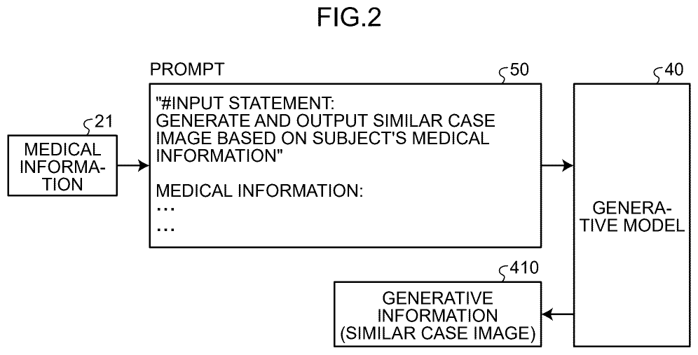

FIG. 2 is a diagram illustrating an exemplary input to and an exemplary output from the generative model 40 according to the first embodiment. As illustrated in FIG. 2, the prompt 50 includes, for example, text data “generate and output a similar case image based on subject's medical information” input by the user, and information indicating a location where the medical information to be referred to is saved.

The subject's (patient's) medical information includes, but is not limited to, the patient's electronic medical record, clinical information, results of medical interviews, test data, medical image data, and diagnostic results. The location where the medical information to be referred to is saved may be a path indicating a save location in the medical information system 300 or a path indicating a save location in the memory 120 of the first information processing apparatus 100. The medical information may be directly written in the prompt 50. The types of medical image data included in the medical information include, but not particularly limited to, X-ray image data, X-ray computed tomography (CT) image data, magnetic resonance image data, and ultrasound image data. The expression “subject's medical information” is used in FIG. 2 for the purpose of explanation, but in practice, the prompt 50 may include identification information such as a patient ID that identifies the subject of interest.

The prompt 50 may include image data as well as text data.

The content of the prompt 50 illustrated in FIG. 2 is an example and is not limited to this. As another specific example, the prompt 50 may include medical image data obtained by capturing a non-contrast image of a subject or a path of its save location, and text data to provide an instruction to generate contrast-enhanced medical image data based on the saved medical image data. Alternatively, the prompt 50 may include medical image data that includes the heart in an imaging range or a path of its save location, and text data to provide an instruction to generate medical image data in a phase different from the phase in which the saved medical image data is actually captured. More specifically, the prompt 50 may include medical image data obtained by imaging the heart in diastole (ED) or a path of its save location, and text data to provide an instruction to generate image data that visualizes the heart in systole (ES) based on the saved medical image data. The prompt 50 may include medical image data obtained by imaging the patient's chest or a path of its save location, and text data to provide an instruction to generate medical image data at a breathing timing different from that for the saved medical image data. The prompt 50 may include medical image data obtained by imaging a patient under a certain imaging condition or a path of its save location, and text data to provide an instruction to generate medical image data under an imaging condition different from that for the saved medical image data. The prompt 50 may include medical image data obtained by imaging a patient by a certain modality or a path of its save location, and text data to provide an instruction to generate medical image data corresponding to imaging by a modality different from that of the saved medical image data.

In this way, the generative model 40 generates image data without performing actual imaging, thereby reducing the patient's exposure dose compared with when a scan is performed multiple times using X-ray CT. There are advantages, such as shorter imaging time and less burden on the patient, even compared with imaging with modalities that do not involve radiation exposure.

Returning to FIG. 1, the first acquisition function 153 acquires medical information from the medical information system 300 via the network 400 and the NW interface 110. For example, the first acquisition function 153 may acquire medical information to be used in the prompt 50 from the medical information system 300, based on a user operation. Alternatively, the first acquisition function 153 may acquire predetermined medical information from the medical information system 300 and save the acquired medical information in the memory 120 in advance. The first acquisition function 153 may be referred to as medical information acquisition function (medical information acquisition unit).

The medical information acquired by the first acquisition function 153 is used so as to be included in the prompt 50 or referred to in the prompt 50, as illustrated in FIG. 2. In the example illustrated in FIG. 2, medical information 21 about a patient for whom a similar case image is generated is used.

Returning to FIG. 1, the second acquisition function 154 inputs the prompt 50 generated by the prompt generating function 152 to the generative model 40 and acquires the generative information 410 from the generative model 40. The second acquisition function 154 may be referred to as generative model control function (generative model control unit).

In the example illustrated in FIG. 2, upon receiving an input of the prompt 50, the generative model 40 outputs the generative information 410. The prompt 50 in FIG. 2 includes an instruction “generate and output a similar case image based on subject's medical information”. Therefore, the generative information 410 is a similar case image. In the present embodiment, the image data may be simply expressed as “image”, such as a similar case image in FIG. 2.

The second acquisition function 154 restores (reproduces) the generative information 410 by re-inputting the prompt 50 stored as storage target data in the memory 120 to the generative model 40, based on a user operation received by the receiving function 151. For example, suppose that the prompt 50 generated by the generative model 40 is stored as storage target data in the memory 120 by the storage processing function 155 described later, and that the generative information 410 is not stored as storage target data. Thereafter, when the user desires to check the generative information 410, the user executes an operation to re-input the prompt 50 stored as storage target data in the memory 120 to the generative model 40. As a result, the generative model 40 generates the generative information 410 again from the prompt 50 stored as storage target data in the memory 120. Such regeneration of the generative information 410 by the same prompt 50 is referred to as restoring or reproducing of the generative information 410. When the generative information 410 restored based on the prompt 50 stored as storage target data in the memory 120 is distinguished from the generative information 410 first generated, it is referred to as “restored generative information”.

Returning to FIG. 1, the storage processing function 155 stores, as storage target data in the memory 120, the prompt 50 out of the generative information 410 and the prompt 50 corresponding to the generative information 410. For example, when the prompt 50 is input to the generative model 40, the storage processing function 155 stores the prompt 50 as storage target data in the memory 120. In other words, the storage processing function 155 allows the prompt 50 used for generating the generative information 410 to be saved for a long term.

When the generative information 410 is specified as a storage target by a user operation, the storage processing function 155 stores the generative information 410 as storage target data in the memory 120. The user's operation to specify a storage target is executed on a display screen displayed on the display 140 by the display control function 156 described later.

For example, the storage processing function 155 may store all prompts 50 input to the generative model 40 as storage target data in the memory 120. Alternatively, for example, when the user edits the prompt 50 and inputs the prompt 50 to the generative model 40 multiple times by trial and error, the storage processing function 155 may store the last prompt 50 input to the generative model 40 as storage target data in the memory 120. The storage processing function 155 automatically stores the prompt 50 as storage target data in the memory 120, regardless of user operation. In other words, the storage processing function 155 preferentially stores the prompt 50 out of the generative information 410 and the prompt 50 corresponding to the generative information 410, as storage target data in the memory 120.

The storage processing function 155 stores the generative information 410 as storage target data in the memory 120 in accordance with a user operation to specify the generative information 410 as a storage target. If a user operation to specify the generative information 410 as a storage target is not received, the storage processing function 155 does not store the generative information 410 as storage target data in the memory 120. In other words, the storage processing function 155 stores the generative information 410 as storage target data in the memory 120 only when a user operation to specify the generative information 410 as a storage target is received. Temporary storage, which is needed to perform processing such as displaying the generative information 410 on the display 140, is not particularly restricted, because such temporary storage is not the storage as storage target data.

Since the prompt 50 is stored as storage target data in the memory 120 even when the generative information 410 is not stored as storage target data in the memory 120, the user can obtain the generative information 410 again by re-inputting the prompt 50 to the generative model 40 as needed. In the present embodiment, therefore, it is not essential to set the generative information 410 as storage target data, and the storage processing function 155 sets the generative information 410 as storage target data only when there is an instruction by the user.

The display control function 156 displays the generative information 410 on the display 140. The display control function 156 displays, on the display 140, an image button that receives a user operation to specify whether one or both of the generative information 410 and the prompt 50 corresponding to the generative information 410 are taken as a storage target.

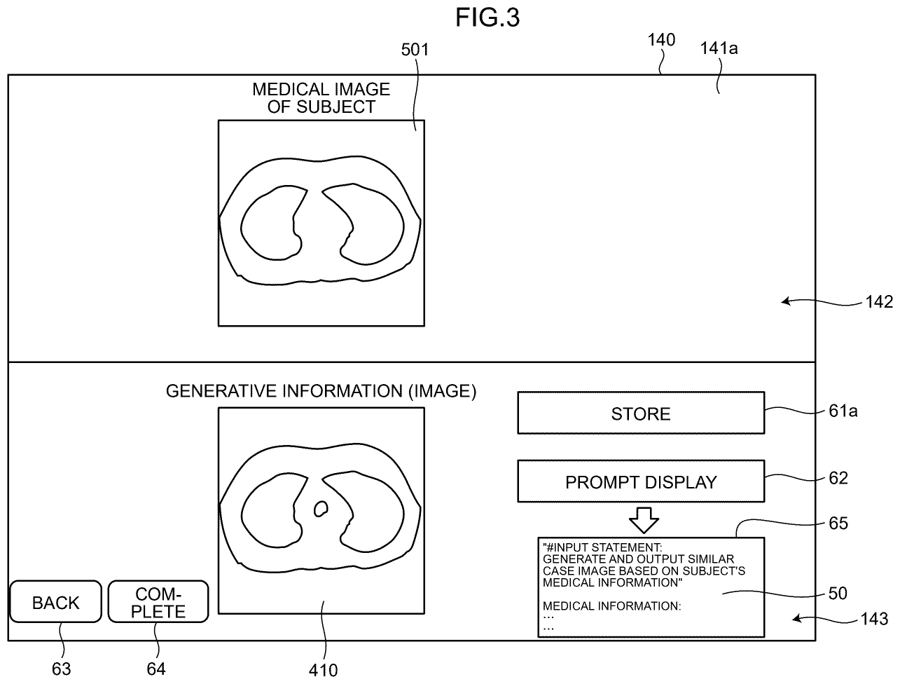

FIG. 3 is a diagram illustrating an exemplary display screen 141a according to the first embodiment. The display screen 141a is displayed on the display 140 by the display control function 156.

As illustrated in FIG. 3, the display screen 141a includes, for example, a medical image 501 of a subject referred to by the prompt 50, the generative information 410, a store button 61a, and a prompt display button 62. As illustrated in FIG. 3, the display screen 141a may include a back button 63, a complete button 64, and the like.

In the example illustrated in FIG. 3, the display screen 141a includes a first screen region 142 that displays the medical image 501, and a second screen region 143 that displays the generative information 410, the store button 61a, the prompt display button 62, the back button 63, and the complete button 64. The screen layout of the display screen 141a is not limited to the example illustrated in FIG. 3.

In FIG. 3, the generative information 410 is a similar case image, but the content of the generative information 410 varies with the content of the prompt 50 and is not limited to the example in FIG. 3.

The store button 61a, the prompt display button 62, the back button 63, and the complete button 64 are image buttons that can receive a user operation.

The store button 61a receives a user operation to specify the generative information 410 as a storage target. For example, when the user operates the input interface 130 such as a mouse and performs an operation of pressing the store button 61a appearing on the display 140, the receiving function 151 described above receives the operation. In this case, the storage processing function 155 stores the generative information 410 appearing on the display screen 141a as storage target data in the memory 120.

When the store button 61a is not pressed by the user, the storage processing function 155 does not store the generative information 410 as storage target data in the memory 120.

The prompt display button 62 receives a user operation to display the prompt 50 used for generating the generative information 410 appearing on the display screen 141a. For example, the display control function 156 does not display the prompt 50 on the display screen 141a in an initial state, but displays a prompt display box 65 as illustrated in FIG. 3 when the prompt display button 62 is pressed by the user. The prompt 50 is displayed in the prompt display box 65. The prompt display box 65 may be provided within the display screen 141a or may be displayed as a separate screen superimposed on the display screen 141a.

The prompt display box 65 may allow the user to edit the prompt 50.

The back button 63 receives a user operation to return from the display screen 141a to an input screen for the prompt 50. For example, when the user presses the back button 63, the display control function 156 transitions the screen to the input screen for the prompt 50. In the input screen for the prompt 50, editing of the prompt 50 and inputting of the edited prompt 50 to the generative model 40 can be executed. When the user executes inputting of the edited prompt 50 to the generative model 40, the display control function 156 displays the generative information 410 generated based on the edited prompt 50 on the display screen 141a.

The complete button 64 receives a user's operation to close the display screen 141a.

The storage processing function 155 does not have to store the prompt 50 as storage target data in the memory 120 when the user presses the back button 63. For example, the user may press the back button 63 to return to the input screen for the prompt 50 to edit the prompt 50 again and re-input the edited prompt 50 to the generative model 40 until the user obtains the generative information 410 as desired. Thus, the storage processing function 155 does not store the prompt 50 as storage target data in the memory 120 when the user presses the back button 63, but when the user finishes viewing the display screen 141a and presses the complete button 64, the storage processing function 155 may store the most recent prompt 50 at the time of pressing as storage target data in the memory 120. When the user presses the store button 61a, the storage processing function 155 may store the prompt 50 corresponding to the generative information 410 appearing on the display screen 141a as storage target data in the memory 120. Alternatively, the storage processing function 155 may store all the prompts 50 input to the generative model 40 as storage target data in the memory 120.

Returning to FIG. 1, the deletion processing function 157 deletes the storage target data stored in the memory 120 when a predetermined deletion condition is met or when a deletion operation is executed by the user. In the present embodiment, only the generative information 410 is a target to be deleted based on the deletion condition, and the prompt 50 is not deleted.

The predetermined deletion condition is, for example, that a predetermined period of time passes since the storage target data is stored, the remaining data capacity of a storage area for storage target data in the memory 120, and that there are no user views for a predetermined period of time. User views of the generative information 410 may be counted based on, for example, the user's operation log or access log. The length of the predetermined period of time may be, for example, about 1-year, or may be determined as desired by an administrator. The predetermined deletion condition is not limited to the above examples.

The deletion processing function 157 may determine the storage target data to be deleted, based on a combination of plural deletion conditions. The deletion conditions may be set by logical operations. The deletion conditions may be determined based on predefined conditional expressions using various logical operators (logical sum (OR operation), logical product (AND operation), exclusive OR (XOR operation), and NOT (NOT operation)), or various logical operators may be presented to the user so that the user can set conditional expressions corresponding to the deletion conditions. Alternatively, only one deletion condition may be set. The deletion condition(s) may be set by, for example, the administrator of the first information processing apparatus 100.

The procedure of a process executed in the first information processing apparatus 100 configured as described above will now be described.

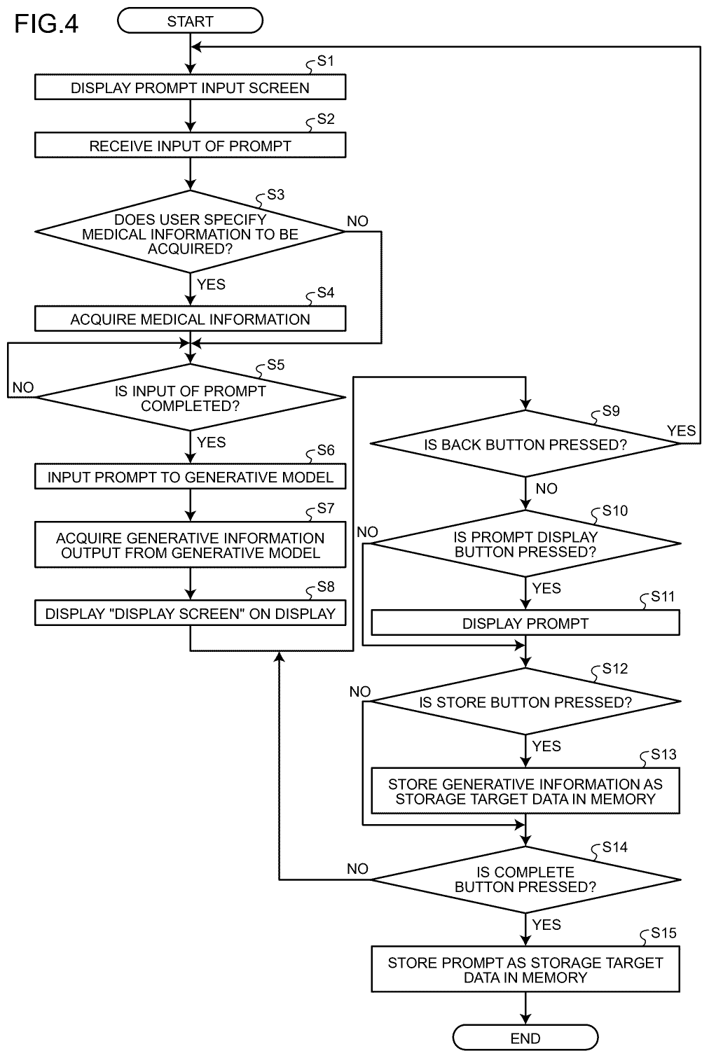

FIG. 4 is a flowchart illustrating an exemplary procedure of a process related to generation and saving of generative information 410 according to the first embodiment. The process in this flowchart is executed when, for example, an operation to open the input screen for the prompt 50 is performed by the user.

First, the display control function 156 displays the prompt input screen on the display 140 (S1).

The receiving function 151 then receives an input of the prompt 50 by the user (S2).

If the user specifies the medical information 21 to be acquired for use in creating the prompt 50 (Yes at S3), the first acquisition function 153 acquires the specified medical information 21 from the medical information system 300 (S4).

The user may generate the prompt 50 without using the medical information 21 in the medical information system 300. If the user does not specify the medical information 21 to be acquired for use in creating the prompt 50 (No at S3), the process at S4 is not executed and the process proceeds to S5. If the generative model 40 in the second information processing apparatus 200 can acquire the specified medical information 21 from the medical information system 300, the prompt 50 may include a path indicating a save location in the medical information system 300, rather than the medical information 21 itself. In this case, the first acquisition function 153 does not have to download the medical information 21 from the medical information system 300 to the first information processing apparatus 100.

If the input of the prompt 50 by the user is completed (Yes at S5), the prompt generating function 152 generates the prompt 50 based on the user's input. The second acquisition function 154 then inputs the generated prompt 50 to the generative model 40 (S6). The generative model 40 generates the generative information 410, based on the input prompt 50. While the input of the prompt 50 by the user is not completed (No at S5), the process at S6 is not executed and the process waits.

The second acquisition function 154 then acquires the generative information 410 output from the generative model 40 (S7).

The display control function 156 then displays the display screen 141a including the acquired generative information 410 on the display 140, as illustrated in FIG. 3 (S8). The process at S9 to S15 is executed based on various operations on the display screen 141a by the user.

For example, if the back button 63 is pressed by the user on the display screen 141a (Yes at S9), the display control function 156 transitions the screen on the display 140 to the prompt input screen, and the process returns to S1. If S1 is executed twice or more, the input screen with the prompt 50 previously input being displayed may appear. If the back button 63 is not pressed (No at S9), the display control function 156 continues to display the display screen 141a.

If the prompt display button 62 is pressed by the user (Yes at S10), the display control function 156 displays the prompt display box 65 including the prompt 50 (S11). If the prompt display button 62 is not pressed (No at S10), the display control function 156 does not display the prompt 50.

If the store button 61a is pressed by the user (Yes at S12), the storage processing function 155 stores the generative information 410 appearing on the display screen 141a as storage target data in the memory 120 (S13). If the store button 61a is not pressed (No at S12), the storage processing function 155 does not store the generative information 410 at least as storage target data in the memory 120.

Even if the back button 63 is pressed after the store button 61a is pressed by the user, the storage processing function 155 saves the prompt 50 and the generative information 410 as storage target data in the memory 120.

The process at S9 to S14 is repeated until the complete button 64 is pressed by the user (No at S14).

If the complete button 64 is pressed by the user (Yes at S14), the storage processing function 155 stores the prompt 50 corresponding to the generative information 410 appearing on the display screen 141a, as storage target data in the memory 120 (S15). The display control function 156 may close the display screen 141a. Here, the process in this flowchart ends.

The procedure of a process of deleting storage target data, which is executed by the first information processing apparatus 100, will now be described.

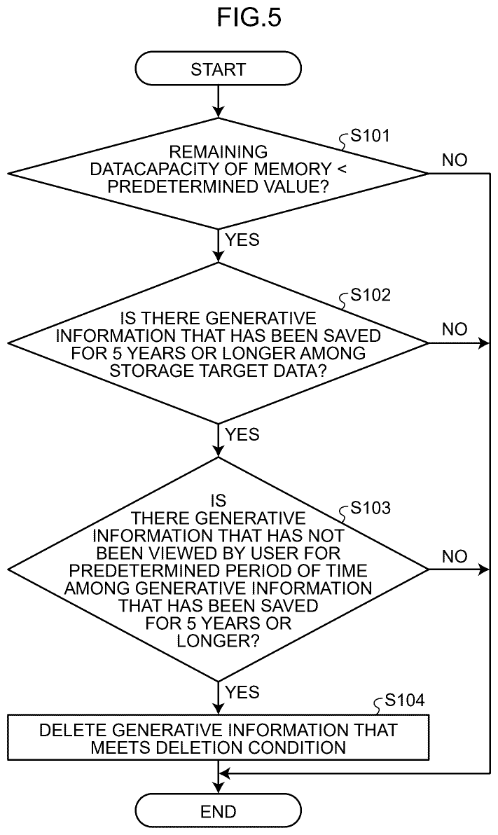

FIG. 5 is a flowchart illustrating an exemplary procedure of the process of deleting storage target data based on a deletion condition according to the first embodiment. The process in this flowchart may be a scheduled process periodically executed or may be executed by a user operation.

First, the deletion processing function 157 determines whether the remaining data capacity of the storage area for storage target data in the memory 120 is less than a predetermined value. If the remaining data capacity of the storage area for storage target data in the memory 120 is equal to or greater than the predetermined value (No at S101), the deletion process is not executed and the process in this flowchart ends.

If the remaining data capacity of the storage area for storage target data in the memory 120 is less than the predetermined value (Yes at S101), the deletion processing function 157 determines whether there is any generative information 410 that has been saved for 5-years or longer, among the storage target data saved in the memory 120. If there is no generative information 410 that has been saved for 5-years or longer (No at S102), the deletion process is not executed and the process in this flowchart ends. In this case, the deletion processing function 157 may output a warning indicating that the remaining data capacity of the storage area for storage target data is getting low.

If there is any generative information 410 that has not been viewed by the user for a predetermined period of time, among the generative information 410 that has been saved for 5-years or longer (Yes at S103), the deletion processing function 157 deletes the generative information 410 as generative information 410 that meets the deletion condition (S104). As used herein, “not viewed by the user for a predetermined period of time” means not viewed by the user for a predetermined period of time in the past from the point in time of the process in FIG. 5.

If there is no generative information 410 that has not been viewed by the user for a predetermined period of time, among the generative information 410 that has been saved for 5-years or longer (No at S103), the deletion process is not executed and the process in this flowchart ends. Also in this case, the deletion processing function 157 may output a warning or the like indicating that the remaining data capacity of the storage area for storage target data is getting low. Here, the process in this flowchart ends.

In the flowchart in FIG. 5, the generative information 410 is a target to be deleted when all the following deletion conditions are met: “the remaining data capacity of the storage area for storage target data in the memory 120 is less than a predetermined value”, “saved for 5-years or longer”, and “not viewed by the user for a predetermined period of time”. However, the contents and combinations of deletion conditions are not limited to these.

In addition to the process based on the deletion conditions illustrated in FIG. 5, any storage target data can be deleted based on, for example, an operation by a user with administrative authority.

In this way, the first information processing apparatus 100 in the present embodiment inputs the generated prompt 50 to the generative model 40 and acquires the generative information 410 from the generative model 40. The first information processing apparatus 100 then stores the prompt 50 out of the acquired generative information 410 and the prompt 50 corresponding to the generative information 410, as storage target data in the memory 120. The first information processing apparatus 100 in the present embodiment therefore can reduce increase in the amount of saved data required for use of the generative model 40, compared with when all pieces of the generated generative information 410 are targets to be saved for a long term. By storing the prompt 50 as storage target data in the memory 120, the first information processing apparatus 100 can obtain the generative information 410 again even if the generative information 410 is not saved by, for example, the user re-input of the prompt 50 to the generative model 40 as needed.

When the prompt 50 is input to the generative model 40, the first information processing apparatus 100 in the present embodiment stores the prompt 50 as storage target data in the memory 120. The first information processing apparatus 100 in the present embodiment therefore can automatically save the prompt 50 used for generating the generative information 410 for a long term, thereby reducing the occurrence of unintentional failure to save the prompt 50.

The first information processing apparatus 100 in the present embodiment receives a user operation to specify the generative information 410 as a storage target, and stores the generative information 410 as storage target data in the memory 120 in accordance with the operation. The first information processing apparatus 100 in the present embodiment stores the prompt 50 as storage target data in the memory 120 regardless of user operation. The first information processing apparatus 100 in the present embodiment therefore can reduce increase in the amount of saved data by basically setting only the prompt 50 as a storage target, and can additionally store the generative information 410 as storage target data in the memory 120 when there is an explicit instruction by the user.

The first information processing apparatus 100 in the present embodiment displays the store button 61a, which is an image button that can receive a user operation to specify the generative information 410 as a storage target, together with the generative information 410 on the display 140. Therefore, the user can operate the store button 61a while checking the target generative information 410.

The first information processing apparatus 100 in the present embodiment includes the memory 120, which is a non-volatile storage device. The prompt 50 and the generative information 410 stored as storage target data in the memory 120 are not deleted except when a predetermined deletion condition is met or when a deletion operation is executed by the user. The first information processing apparatus 100 in the present embodiment therefore can appropriately save data that needs to be saved for a long term in the business in which the first information processing apparatus 100 is used. The storage target data is data that is saved by the first information processing apparatus 100 for a long term, and the first information processing apparatus 100 in the present embodiment can restrict targets of the storage target data as described above, thereby reducing increase in the amount of saved data required for use of the generative model 40.

The generative information 410 that the first information processing apparatus 100 in the present embodiment allows the generative model 40 to generate includes image data. In general, when the generative information 410 contains image data and the prompt 50 is text, the data volume of the generative information 410 is significantly larger than the data volume of the prompt 50. By preferentially storing the prompt 50 as storage target data in the memory 120 out of the generative information 410 and the prompt 50, it is possible to effectively reduce the increase in the amount of saved data.

### Second Embodiment

In the first embodiment described above, the first information processing apparatus 100 stores the prompt 50 as storage target data in the memory 120, regardless of user operation, and determines, only for the generative information 410, whether to set the prompt 50 as storage target data in accordance with a user operation. In contrast, in the present embodiment, whether to store both the prompt 50 and the generative information 410 as storage target data is determined in accordance with a user operation.

The information processing system S in the present embodiment includes, for example, the first information processing apparatus 100, the second information processing apparatus 200, and the medical information system 300 in the same manner as in the first embodiment illustrated in FIG. 1. The first information processing apparatus 100 in the present embodiment includes the NW interface 110, the memory 120, the input interface 130, the display 140, and the processing circuitry 150, in the same manner as in the first embodiment. A configuration similar to that of the first embodiment is denoted by the same symbol and will not be further elaborated.

The processing circuitry 150 in the present embodiment includes the receiving function 151, the prompt generating function 152, the first acquisition function 153, the second acquisition function 154, the storage processing function 155, the display control function 156, and the deletion processing function 157, in the same manner as in the first embodiment.

The receiving function 151, the prompt generating function 152, the first acquisition function 153, the second acquisition function 154, and the deletion processing function 157 have functions similar to those of the first embodiment.

In addition to the functions in the first embodiment, the display control function 156 in the present embodiment displays, on the display 140, an image button that receives a user operation to specify whether one or both of the generative information 410 and the prompt 50 corresponding to the generative information 410 are set as a storage target.

The storage processing function 155 in the present embodiment stores one or both of the generative information 410 and the prompt 50 corresponding to the generative information 410 as storage target data in the memory 120 in accordance with a user operation.

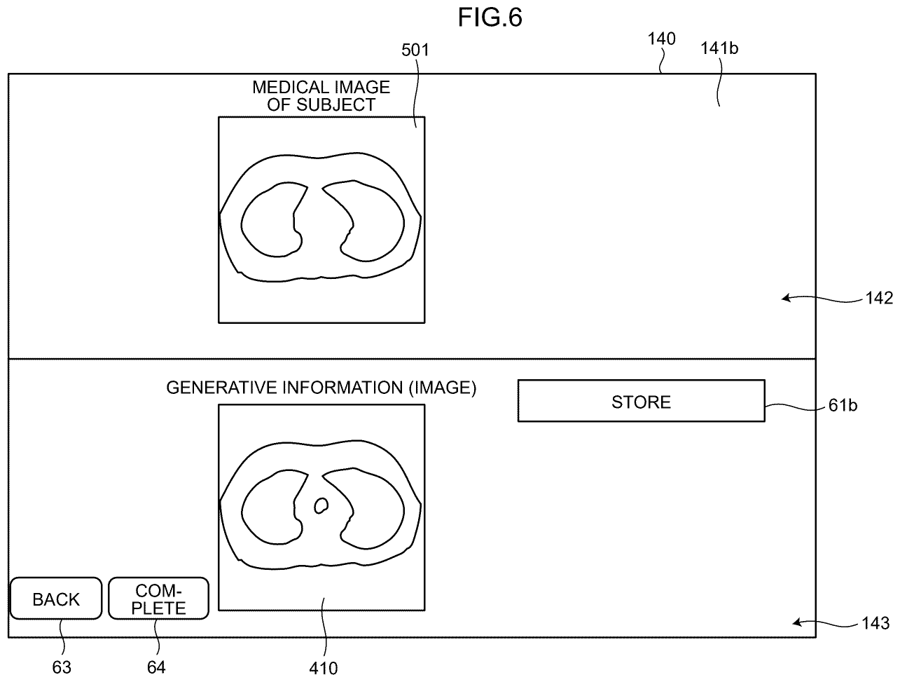

FIG. 6 is a diagram illustrating an exemplary display screen 141b according to the second embodiment. As illustrated in FIG. 6, the display screen 141b includes, for example, the medical image 501 of a subject referred to by the prompt 50, the generative information 410, a store button 61b, the back button 63, and the complete button 64. The display screen 141b may include the prompt display button 62 in the same manner as in the display screen 141a in the first embodiment. The back button 63 and the complete button 64 have functions similar to those of the first embodiment.

When the store button 61b is pressed by the user, the display control function 156 displays, on the display 140, an image button that receives a user operation to specify whether one or both of the generative information 410 and the prompt 50 corresponding to the generative information 410 are set as a storage target.

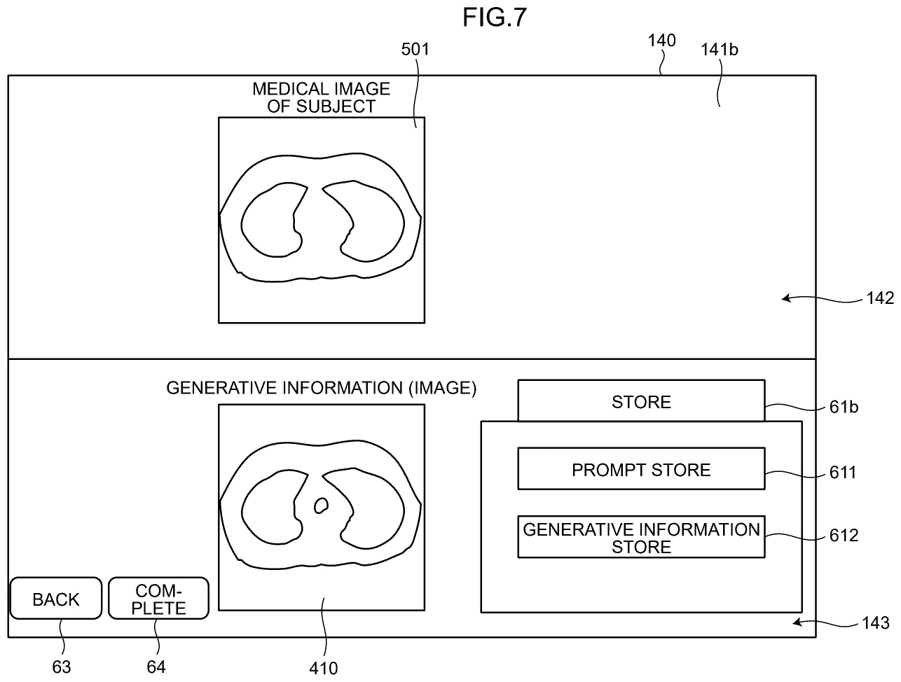

FIG. 7 is a diagram illustrating an exemplary state in which the store button 61b is pressed by the user in FIG. 6. As illustrated in FIG. 7, when the store button 61b is pressed by the user, the display control function 156 displays icons of a prompt store button 611 and a generative information store button 612 on the display 140. The prompt store button 611 and the generative information store button 612 may be displayed within the display screen 141b or may be displayed on a separate screen superimposed on the display screen 141b.

When the prompt store button 611 is pressed by the user, the storage processing function 155 stores the prompt 50 corresponding to the generative information 410 appearing on the display screen 141b as storage target data in the memory 120. When the prompt store button 611 is not pressed by the user, the storage processing function 155 does not store the prompt 50 corresponding to the generative information 410 appearing on the display screen 141b as storage target data in the memory 120.

When the generative information store button 612 is pressed by the user, the storage processing function 155 stores the generative information 410 appearing on the display screen 141b as storage target data in the memory 120. When the generative information store button 612 is not pressed by the user, the storage processing function 155 does not store the generative information 410 appearing on the display screen 141b as storage target data in the memory 120.

In other words, the icons of the prompt store button 611 and the generative information store button 612 receive a user operation to select selection target data. The prompt store button 611 and the generative information store button 612 therefore are an example of a selector. The method of selection by the user is not limited to the prompt store button 611 and the generative information store button 612. For example, another operation image such as a list box or an operation by a command may be employed.

In this way, the first information processing apparatus 100 in the present embodiment displays, on the display 140, an image button that receives a user operation to specify whether one or both of the generative information 410 and the prompt 50 corresponding to the generative information 410 are set as a storage target, and stores one or both of the generative information 410 and the prompt 50 corresponding to the generative information 410 as storage target data in the memory 120 in accordance with the user operation. The first information processing apparatus 100 in the present embodiment therefore can further reduce increase in the amount of saved data, in addition to the effects of the first embodiment.

### Third Embodiment

In the first and second embodiments described above, a single piece of generative information 410 is output from the generative model 40. In the present third embodiment, when multiple pieces of generative information 410 are output from the generative model 40, a user operation to specify the generative information 410 as a storage target is received for each piece of generative information 410 output from the generative model 40.

The information processing system S in the present embodiment includes, for example, the first information processing apparatus 100, the second information processing apparatus 200, and the medical information system 300 in the same manner as in the first embodiment illustrated in FIG. 1. The generative model 40 in the second information processing apparatus 200 in the present embodiment generates and outputs multiple pieces of generative information 410. The generative information 410 in the present embodiment includes image data. The first information processing apparatus 100 in the present embodiment includes the NW interface 110, the memory 120, the input interface 130, the display 140, and the processing circuitry 150, in the same manner as in the first embodiment. A configuration similar to that of the first embodiment is denoted by the same symbol and will not be further elaborated.

The processing circuitry 150 in the present embodiment includes the receiving function 151, the prompt generating function 152, the first acquisition function 153, the second acquisition function 154, the storage processing function 155, the display control function 156, and the deletion processing function 157, in the same manner as in the first embodiment.

The receiving function 151, the prompt generating function 152, the first acquisition function 153, the second acquisition function 154, and the deletion processing function 157 have functions similar to those of the first embodiment.

In addition to the functions in the first embodiment, the display control function 156 in the present embodiment displays an image button, for each piece of generative information 410 output from the generative model 40, that receives a user operation to specify the generative information 410 as a storage target, on the display 140.

More specifically, the generative model 40 in the present embodiment generates and outputs multiple pieces of image data, based on the input prompt 50. The display control function 156 displays an image button, for each of the multiple pieces of image data output from the generative model 40, that receives a user operation to select the image data as a storage target, on the display 140.

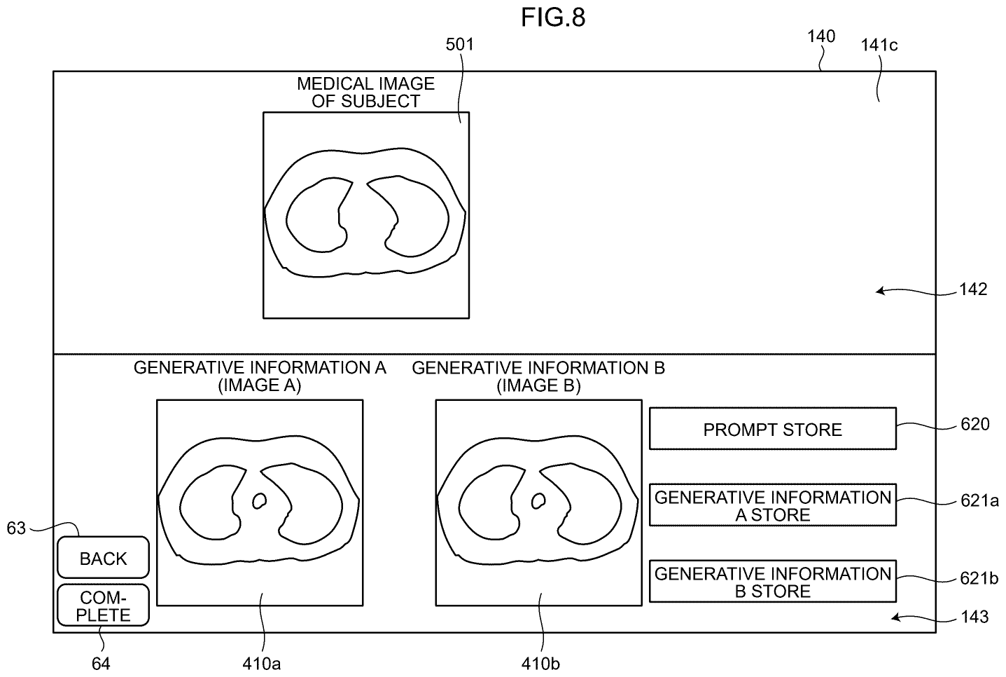

FIG. 8 is a diagram illustrating an exemplary display screen 141c according to the third embodiment. As illustrated in FIG. 8, the display screen 141c includes, for example, the medical image 501 of a subject referred to by the prompt 50, pieces of generative information 410a and 410b, and icons of a prompt store button 620, a generative information A store button 621a, a generative information B store button 621b, the back button 63, and the complete button 64. The prompt store button 620 is not necessarily displayed. The storage processing function 155 may store the corresponding prompt 50 in the memory 120, in the same manner as in the first embodiment, when the user selects the generative information A store button 621a or the generative information B store button 621b. The storage processing function 155 may control the memory 120 to store the prompt 50 when the user selects the complete button 64, regardless of whether the generative information A store button 621a and the generative information B store button 621b are pressed. The display screen 141c may include the prompt display button 62 in the same manner as in the display screen 141a in the first embodiment. The back button 63 and the complete button 64 have functions similar to those of the first embodiment. The prompt store button 620 has a function similar to that of the prompt store button 611 in the second embodiment.

The multiple pieces of generative information 410a and 410b are both image data (images A and B).

The icon of the generative information A store button 621a is an image button corresponding to the generative information 410a (generative information A (image A)). The icon of the generative information B store button 621b is an image button corresponding to the generative information 410b (generative information B (image B)). Hereafter, the generative information A store button 621a and the generative information B store button 621b are simply referred to as generative information store button 621 when they are not specifically distinguished from each other. The generative information store button 621 is an example of a selector.

In FIG. 8, two pieces of generative information 410a and 410b are output from the generative model 40. However, three or more pieces of generative information 410 may be output. The display control function 156 displays the generative information store buttons 621 corresponding to respective pieces of output image data, in accordance with the number of pieces of image data output from the generative model 40. In other words, the display control function 156 dynamically generates the generative information store button(s) 621 in accordance with the number of pieces of image data output from the generative model 40.

When the generative information A store button 621a is pressed by the user, the storage processing function 155 stores the generative information 410a appearing on the display screen 141c as storage target data in the memory 120. When the generative information A store button 621a is not pressed by the user, the storage processing function 155 does not store the generative information 410a as storage target data in the memory 120.

When the generative information B store button 621b is pressed by the user, the storage processing function 155 stores the generative information 410b appearing on the display screen 141c as storage target data in the memory 120. When the generative information B store button 621b is not pressed by the user, the storage processing function 155 does not store the generative information 410b as storage target data in the memory 120.

The method of selection by the user is not limited to the prompt store button 620 and the generative information store button 621. For example, another operation image such as a list box or an operation by a command may be employed.

In this way, the first information processing apparatus 100 in the present embodiment displays an image button, for each piece of image data output from the generative model 40, that receives a user operation to specify the image data as a storage target, on the display 140. In the first information processing apparatus 100 in the present embodiment, therefore, when multiple pieces of image data are included in the generative information 410, the user can individually specify whether to save the data for a long term. This configuration can effectively reduce increase in the amount of saved data, compared with when all pieces of image data are uniformly set as storage targets.

### Fourth Embodiment

In the first to third embodiments described above, the first information processing apparatus 100 determines storage target data in accordance with a user operation. In the present fourth embodiment, the first information processing apparatus 100 evaluates the necessity to store the generative information 410 and determines whether to set the generative information 410 as storage target data based on the evaluation result.

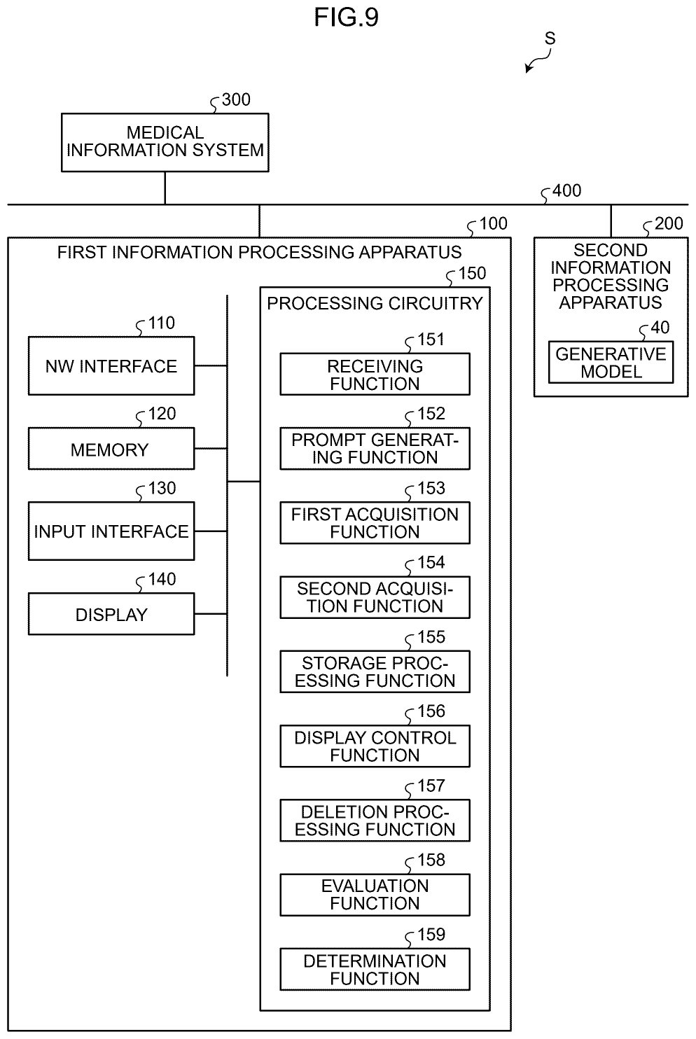

FIG. 9 is a diagram illustrating an exemplary overall configuration of the information processing system S according to the fourth embodiment. The information processing system S in the present embodiment includes, for example, the first information processing apparatus 100, the second information processing apparatus 200, and the medical information system 300 in the same manner as in the first embodiment. The first information processing apparatus 100 in the present embodiment includes the NW interface 110, the memory 120, the input interface 130, the display 140, and the processing circuitry 150, in the same manner as in the first embodiment. A configuration similar to that of the first embodiment is denoted by the same symbol and will not be further elaborated.

The processing circuitry 150 in the present embodiment includes the receiving function 151, the prompt generating function 152, the first acquisition function 153, the second acquisition function 154, the storage processing function 155, the display control function 156, the deletion processing function 157, an evaluation function 158, and a determination function 159. The evaluation function 158 is an example of an evaluation unit. The determination function 159 is an example of a determination unit.

The receiving function 151, the prompt generating function 152, the first acquisition function 153, the second acquisition function 154, and the display control function 156 have functions similar to those of the first embodiment.

The evaluation function 158 evaluates the necessity to store the generative information 410. The “necessity to store” is the necessity for the generative information 410 to be saved as storage target data in the memory 120 for a long term. The necessity to store increases as the importance of the generative information 410 increases. In the present embodiment, since the information processing system S is used in the medical field and the generative information 410 includes medical information about a patient, the evaluation function 158 evaluates the clinical importance of the generative information 410. The evaluation criterion for the necessity to store may differ depending on the business field in which the information processing system S is used. The clinical importance is an example of the property of the generative information 410.

For example, with the stronger relevance between the generative information 410 and the diagnostic result for a patient for whom the generative information 410 is referred to during diagnosis, the evaluation function 158 evaluates the clinical importance of the generative information 410 more highly. In other words, with the stronger relevance between the generative information 410 and the diagnostic result for a patient for whom the generative information 410 is referred to during diagnosis, the evaluation function 158 evaluates the necessity to store the generative information 410 as being higher. The degree of relevance to the diagnostic result is stronger when, for example, a symptom of the disease included in the diagnostic result is directly represented in the image data of the generative information 410. With less other medical information 21 that the physician refers to during diagnosis, the evaluation function 158 may evaluate the relevance between the generative information 410 and the diagnostic result as being stronger. The degree of relevance to a diagnostic result may be determined by the generative model 40 or other generative models.

The evaluation function 158 may express the degree of clinical importance as a numerical value or as a level such as “low”, “medium”, or “high”.

The evaluation criterion for the clinical importance of the generative information 410 is not limited to the degree of relevance to the diagnostic result. For example, other examples of the evaluation criterion include “the clinical importance is evaluated as being high when there is no other test data”, “the clinical importance is evaluated as being high when there is less test data other than the generative information 410”, “the clinical importance is evaluated as being high when the number of times (or the checking time, the number of persons who check) the physician checks the generative information 410 at the time of diagnosis”, and “the clinical importance is evaluated as being high when the result contradicts other medical information”.

“Other test data” and “other medical information” are test data and test results that are not included in the prompt 50 and are not referred to by the prompt 50.

As used herein, “the generative information 410 contradicts other medical information 21” means that, for example, a disease state indicated by the generative information 410 is different from a disease state indicated by other test results or captured medical image data. Specifically, this applies when cancer is clearly visualized in image data that is the generative information 410, but a tumor marker test value is not positive. This also applies when the size of tumor visualized in medical image data obtained by imaging a patient by a modality is different from the size of tumor visualized in image data that is the generative information 410. As used herein, “the generative information 410 contradicts other medical information” is not limited to a case where either the generative information 410 or other medical information is incorrect, but also includes a case where the generative information 410 provides information that is not detected in a test or the like. Physicians may derive a diagnostic result from multiple perspectives by referring to multiple pieces of contradictory information during diagnosis. Thus, setting multiple pieces of contradictory information as storage targets may be useful in monitoring the patient's progress or in verification at a later day.

In contrast, the generative information 410 may clearly contain an error, compared with other medical information. Thus, the evaluation criterion may include a criterion “the more likely the generative information 410 contains an error, the lower the clinical importance of the information is evaluated”.

The evaluation function 158 may determine the probability (likelihood) of the generative information 410 and evaluate the clinical importance more highly with the higher likelihood. Any known method can be employed as a method for determining the likelihood of the generative information 410. For example, the evaluation function 158 may use another generative model to determine the likelihood of the generative information 410. The other generative model may be provided in the first information processing apparatus 100 or in an external device. The evaluation function 158 may be a generative model for determining the likelihood of the generative information 410. Alternatively, the generative model 40 may have an automatic evaluation function to determine the likelihood of the generative information 410 generated by itself. In this case, the evaluation function 158 evaluates the clinical importance in accordance with the likelihood determined by the generative model 40.

The clinical importance of the generative information 410 may be determined by a physician. In this case, the evaluation function 158 acquires the clinical importance of the generative information 410 input by the physician.

The evaluation function 158 may combine multiple evaluation criteria to determine the degree of clinical importance. The evaluation criterion may be predetermined, or the evaluation criterion may be selected by an administrator or the like of the first information processing apparatus 100. The evaluation criterion for the clinical importance is not limited to the above examples.

The determination function 159 determines whether to set the generative information 410 as storage target data based on the evaluation result by the evaluation function 158. For example, when the degree of clinical importance of the generative information 410 determined by the evaluation function 158 is equal to or higher than a predetermined standard, the determination function 159 determines to set the generative information 410 as storage target data. When the degree of clinical importance of the generative information 410 is lower than the predetermined standard, the determination function 159 determines not to set the generative information 410 as storage target data. The predetermined standard may be set in advance or may be set by the administrator of the first information processing apparatus 100.

In addition to the functions in the first embodiment, the storage processing function 155 in the present embodiment stores the prompt 50, or both the generative information 410 and the prompt 50, as storage target data in the memory 120, based on the determination result by the determination function 159.

More specifically, when the determination function 159 determines to set the generative information 410 as storage target data, the storage processing function 155 stores the generative information 410 and the prompt 50 corresponding to the generative information 410 as storage target data in the memory 120. When the determination function 159 determines not to set the generative information 410 as storage target data, the storage processing function 155 stores the prompt 50 as storage target data in the memory 120, but does not store the generative information 410 as storage target data in the memory 120.

In the present embodiment, the storage processing function 155 stores the prompt 50 as storage target data in the memory 120, regardless of the determination result. The evaluation function 158 may have the function to evaluate the necessity to store even for the prompt 50. The evaluation criterion for evaluating the necessity to store the prompt 50 may be different from the evaluation criterion for evaluating the necessity to store the generative information 410.

In addition to the functions in the first embodiment, the deletion processing function 157 in the present embodiment may use, as a predetermined deletion condition, a criterion similar to the evaluation criterion used by the evaluation function 158 described above or the evaluation result by the evaluation function 158. For example, the predetermined deletion condition may include the degree of relevance between the generative information 410 and the diagnostic result, or the evaluation result for the clinical importance being lower than a predetermined standard.

In this way, the first information processing apparatus 100 in the present embodiment evaluates the necessity to store the generative information 410 and determines whether to set the generative information 410 as storage target data based on the evaluation result. The first information processing apparatus 100 in the present embodiment stores the prompt 50, or both the generative information 410 and the prompt 50, as storage target data in the memory 120, based on the determination result. The first information processing apparatus 100 in the present embodiment therefore can automatically set the generative information 410 as a storage target in accordance with the necessity to store the generative information 410, in addition to the effects of the first embodiment. This configuration reduces the time and effort required for the user to determine the necessity to store the individual generative information 410. This configuration also reduces the possibility that generative information 410 with higher necessity to store disappears without being stored.

### Fifth Embodiment

In the first to fourth embodiments described above, the first information processing apparatus 100 sets the generative information 410 or the prompt 50 as storage target data. In the present fifth embodiment, the medical information 21 relative to the generative information 410 is also set as storage target data.

The information processing system S in the present embodiment includes, for example, the first information processing apparatus 100, the second information processing apparatus 200, and the medical information system 300 in the same manner as in the first and fourth embodiments illustrated in FIGS. 1 and 9. The first information processing apparatus 100 in the present embodiment includes the NW interface 110, the memory 120, the input interface 130, the display 140, and the processing circuitry 150, in the same manner as in the first and fourth embodiments. A configuration similar to that of the first embodiment is denoted by the same symbol and will not be further elaborated.

The processing circuitry 150 in the present embodiment includes the receiving function 151, the prompt generating function 152, the first acquisition function 153, the second acquisition function 154, the storage processing function 155, the display control function 156, the deletion processing function 157, the evaluation function 158, and the determination function 159, in the same manner as in the fourth embodiment.

The receiving function 151, the prompt generating function 152, the second acquisition function 154, the deletion processing function 157, the evaluation function 158, and the determination function 159 have functions similar to those of the fourth embodiment.

In addition to the functions in the fourth embodiment, the evaluation function 158 in the present embodiment evaluates the relevance between various medical information 21 stored in the medical information system 300 and the generative information 410.

As used herein, the medical information 21 with strong relevance to the generative information 410 is, for example, the medical information 21 included in the prompt 50 corresponding to the generative information 410 or the medical information 21 referred to by the prompt 50. Alternatively, the generative model 40 may identify the medical information 21 with strong relevance to the generative information 410. In this case, the evaluation function 158 acquires, from the generative model 40, the result of identifying the medical information 21 with strong relevance to the generative information 410. For example, the generative model 40 may identify the medical information 21 referred to by RAG in generating the generative information 410 as the medical information 21 with strong relevance to the generative information 410.

Alternatively, a condition for the medical information 21 with strong relevance to the generative information 410 may be defined in advance. For example, when it has been decided that the generative model 40 is used for generating the generative information 410 based on medical image data obtained by imaging a patient, medical image data that is the source of the generative information 410, imaging conditions of the modality of the source medical image data, and information on a patient to be imaged may be defined as the medical information 21 with strong relevance to the generative information 410.

The evaluation function 158 may evaluate the degree of relevance between the generative information 410 and the medical information 21 numerically or at two levels of relevance, such as “yes” and “no”.

In addition to the functions in the first and fourth embodiments, the first acquisition function 153 in the present embodiment acquires medical information from the medical information system 300, based on the evaluation result of the relevance to the generative information 410 by the evaluation function 158. For example, the first acquisition function 153 may acquire the medical information 21 evaluated as having stronger relevance to the generative information 410 than a predetermined standard. If the medical information 21 that is an acquisition target has already been acquired for generation of the prompt 50, the re-acquisition process by the first acquisition function 153 may be omitted. When the relevance to the generative information 410 is evaluated at two levels “yes” and “no”, the first acquisition function 153 may set the medical information 21 evaluated as having relevance to the generative information 410 as an acquisition target.

In addition to the functions in the first and fourth embodiments, the storage processing function 155 in the present embodiment stores the medical information 21 as storage target data in the memory 120, based on the evaluation result of the relevance to the generative information 410 by the evaluation function 158. For example, if there is medical information 21 evaluated as having higher relevance to the generative information 410 than a predetermined standard, the storage processing function 155 may store, in the memory 120, the medical information 21 as storage target data and the prompt 50 so as to correlate with each other. When the relevance to the generative information 410 is evaluated at two levels “yes” and “no”, the storage processing function 155 may store the medical information 21 evaluated as having relevance to the generative information 410 as storage target data in the memory 120.

If there is no medical information 21 evaluated as having higher relevance to the generative information 410 than a predetermined standard, or there is no medical information 21 evaluated as having relevance to the generative information 410, the storage processing function 155 may store only the prompt 50 as storage target data, without setting the medical information 21 as a storage target.

In addition to the functions in the first and fourth embodiments, the display control function 156 in the present embodiment displays the medical information 21 on the display 140, based on the evaluation result of the relevance to the generative information 410 by the evaluation function 158. For example, the display control function 156 may display the medical information 21 evaluated as having higher relevance to the generative information 410 than a predetermined standard, or the medical information 21 evaluated as having relevance to the generative information 410, on the display screens 141a to 141c, together with the generative information 410.

In this way, the first information processing apparatus 100 in the present embodiment stores, in the memory 120, the medical information 21 as storage target data and the prompt 50 so as to correlate with each other, based on the evaluation result of the relevance between the generative information 410 and the medical information 21. The first information processing apparatus 100 in the present embodiment therefore can reduce the occurrence of a situation where, for example, the generative information 410 fails to be reproduced because of the absence of medical information 21 to be referred to by the prompt 50 when the prompt 50 is re-input to the generative model 40 at a later day, or the validity of generative information 410 fails to be verified because the relevant medical information 21 is unknown.

### Sixth Embodiment

In the present sixth embodiment, the first information processing apparatus 100 further stores a previous version of the generative model 40.

The information processing system S in the present embodiment includes, for example, the first information processing apparatus 100, the second information processing apparatus 200, and the medical information system 300 in the same manner as in the first embodiment illustrated in FIG. 1. The first information processing apparatus 100 in the present embodiment includes the NW interface 110, the memory 120, the input interface 130, the display 140, and the processing circuitry 150, in the same manner as in the first and fourth embodiments. A configuration similar to that of the first embodiment is denoted by the same symbol and will not be further elaborated.

The processing circuitry 150 in the present embodiment includes the receiving function 151, the prompt generating function 152, the first acquisition function 153, the second acquisition function 154, the storage processing function 155, the display control function 156, and the deletion processing function 157, in the same manner as in the first embodiment.

The receiving function 151, the prompt generating function 152, the first acquisition function 153, the second acquisition function 154, the display control function 156, and the deletion processing function 157 have functions similar to those of the first embodiment.

In addition to the functions in the first embodiment, when storing the prompt 50 as storage target data in the memory 120, the storage processing function 155 in the present embodiment stores version information of the generative model 40, to which the prompt 50 is input, so as to correlate with the prompt 50. The version information of the generative model 40 is, for example, a number indicating the version of the generative model 40.

In general, the version of the generative model 40 is updated periodically. A new version of the generative model 40 may have, for example, parameters updated as a result of additional training performed on a previous version of the generative model 40, or have changes in functions. Different versions of the generative model 40 may output different generative information 410 even when the same prompt 50 is input.

The previous version of the generative model 40 may be stored in the memory 120 of the first information processing apparatus 100 or in the second information processing apparatus 200.

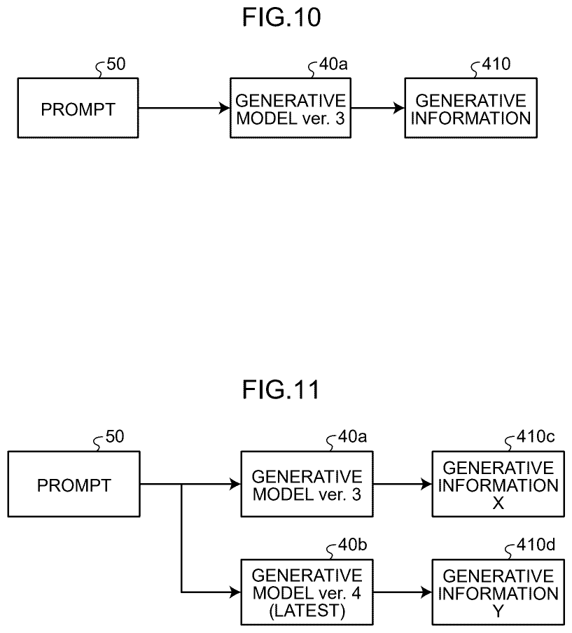

FIG. 10 is a diagram illustrating an exemplary relationship between a version of a generative model 40a and generative information 410 according to the sixth embodiment. For example, suppose that the second acquisition function 154 inputs the prompt 50 to the generative model 40a and obtains the generative information 410. If the version of the generative model 40a at the time of generating this generative information 410 is “ver. 3”, the input of the prompt 50 to a version released later than “ver. 3” does not always result in the same generative information 410.

The storage processing function 155 therefore stores, as storage target data in the memory 120, the version of the generative model 40a (“ver. 3” in the example illustrated in FIG. 10) so as to correlate with the prompt 50. In this case, if the need to reproduce the generative information 410 using the prompt 50 arises at a later day, the generative model 40a of the version that has correlation with this prompt 50 can be identified as a destination to which the prompt 50 is input.

### Seventh Embodiment

In the present seventh embodiment, in addition to the sixth embodiment, when the prompt 50 input to a previous version of the generative model 40 is re-input, the prompt 50 is input to multiple versions of the generative model 40.

The information processing system S in the present embodiment includes, for example, the first information processing apparatus 100, the second information processing apparatus 200, and the medical information system 300 in the same manner as in the first embodiment illustrated in FIG. 1. The first information processing apparatus 100 in the present embodiment includes the NW interface 110, the memory 120, the input interface 130, the display 140, and the processing circuitry 150, in the same manner as in the first and fourth embodiments. A configuration similar to that of the first embodiment is denoted by the same symbol and will not be further elaborated.

The processing circuitry 150 in the present embodiment includes the receiving function 151, the prompt generating function 152, the first acquisition function 153, the second acquisition function 154, the storage processing function 155, the display control function 156, and the deletion processing function 157, in the same manner as in the first embodiment.

The receiving function 151, the prompt generating function 152, the first acquisition function 153, the storage processing function 155, and the deletion processing function 157 have functions similar to those of the first or sixth embodiment.

In addition to functions similar to those of the first and sixth embodiments, the second acquisition function 154 in the present embodiment inputs the prompt 50 to multiple versions of the generative model 40 when the prompt 50 input to a previous version of the generative model 40 is re-input.

FIG. 11 is a diagram illustrating an exemplary relationship between the versions of generative models 40a and 40b and generative information 410c and 410d when the prompt 50 is re-input according to the seventh embodiment. For example, the second acquisition function 154 inputs the prompt 50 to both of the generative model 40a of the version “ver. 3” saved in association with the prompt 50 and the generative model 40b of the latest version “ver. 4”. In this case, the second acquisition function 154 acquires the generative information 410c (generative information X) generated by the generative model 40a and the generative information 410b (generative information Y) generated by the generative model 40b.

In addition to functions similar to those of the first and sixth embodiments, the display control function 156 in the present embodiment displays the generative information 410c and 410d generated by different versions on the display 140 in association with the version information. The display control function 156 may identify and display a difference between the generative information 410c and 410d generated by different versions.

In this way, the first information processing apparatus 100 in the present embodiment can provide the user with the generative information 410 generated by multiple versions of the generative model 40, thereby providing information for making a decision in verifying the contents of the previous generative information 410 or in reconsidering the patient's diagnosis.

The first information processing apparatus 100 in the present embodiment can display the generative information 410c generated by the previous version of the generative model 40a and the generative information 410d generated by the latest version of the generative model 40b in a comparable manner, thereby helping the user to review the previous diagnostic result by taking into account the changes in the generative information 410d generated by the latest version of the generative model 40b.

### Eighth Embodiment

In the present eighth embodiment, the first information processing apparatus 100 inputs one prompt 50 to the same generative model 40 multiple times to obtain multiple pieces of generative information 410.

In general, even if the version of the generative model 40 is the same, the contents of the generative information 410 may change each time the prompt 50 is input. Such changes in the generative information 410 are also referred to as “fluctuations”. Due to the occurrence of such fluctuations, when only the prompt 50 is a storage target and the generative information 410 is not stored, there is an issue of reproducibility of the generative information 410.

The information processing system S in the present embodiment includes, for example, the first information processing apparatus 100, the second information processing apparatus 200, and the medical information system 300 in the same manner as in the first embodiment illustrated in FIG. 1. The first information processing apparatus 100 in the present embodiment includes the NW interface 110, the memory 120, the input interface 130, the display 140, and the processing circuitry 150, in the same manner as in the first embodiment. A configuration similar to that of the first embodiment is denoted by the same symbol and will not be further elaborated.

The processing circuitry 150 in the present embodiment includes the receiving function 151, the prompt generating function 152, the first acquisition function 153, the second acquisition function 154, the storage processing function 155, the display control function 156, and the deletion processing function 157, in the same manner as in the first embodiment.

The receiving function 151, the prompt generating function 152, the first acquisition function 153, and the deletion processing function 157 have functions similar to those of the first or sixth embodiment.

In addition to functions similar to those of the first embodiment, the second acquisition function 154 in the present embodiment inputs the same prompt 50 to the same generative model 40 multiple times to acquire multiple pieces of generative information 410. For example, when the prompt 50 is an instruction to generate similar case image data, the generative model 40 outputs multiple pieces of similar case image data corresponding to the multiple inputs of the prompt 50.

When multiple pieces of generative information 410 include non-image data such as numerical values, the second acquisition function 154 may calculate statistical information of the multiple pieces of generative information 410. The statistical information is, for example, average information and mode information of multiple pieces of generative information 410.

In addition to functions similar to those of the first embodiment, when the same generative information 410 is included in multiple pieces of generative information 410 output from the same generative model 40, the display control function 156 in the present embodiment may employ the largest number of pieces of generative information 410 as a display target.

The display control function 156 may display the statistical information of the multiple pieces of generative information 410 on the display 140. When the generative information 410 is a region image indicating a region such as a lesion in image data, the display control function 156 may display an overlapping region.

The display control function 156 may display a message on the display 140 to provide the user who inputs the prompt 50 with feedback on the magnitude of fluctuations in the multiple pieces of generative information 410.

For example, the second acquisition function 154 may determine the magnitude of fluctuations based on the magnitude of difference in the multiple pieces of generative information 410. In this case, if the fluctuations are greater than a predetermined standard, the display control function 156 may display a message on the display 140 to urge the user to change the prompt 50.

In general, the fluctuations of multiple pieces of generative information 410 are likely to be larger when the content of the prompt 50 is ambiguous or when the prompt 50 is too short and lacks information. The display control function 156 therefore may display, for example, a warning on the display 140 when the prompt 50 stored as storage target data in the memory 120 is ambiguous, resulting in low reproducibility of generative information 410.

The display control function 156 may display a warning on the display 140 when the number of characters in the prompt 50 is equal to or less than a predetermined number of characters, or when the prompt 50 does not meet a predetermined condition. The predetermined number of characters is, for example, fifty (50), but is not limited to this value. The predetermined condition is, for example, a constraint on the generation of the generative information 410. When the prompt 50 contains an instruction to generate image data, the display control function 156 may employ a predetermined condition that the prompt 50 includes the source image data or the path of the save location of the source image data.

The display control function 156 displays the warning when the prompt 50 is stored as storage target data in the memory 120. The warning display may be triggered by, for example, the user pressing the prompt display button 62 on the display screen 141a illustrated in FIG. 3. Alternatively, it may be triggered by the user pressing the complete button 64. When the display control function 156 displays the prompt store button 611, 620 on the display 140 as illustrated in FIGS. 7 and 8, the warning display may be triggered by the user pressing the prompt store button 611, 620.

In addition to functions similar to those of the first embodiment, the storage processing function 155 in the present embodiment may set storage target data based on the magnitude of fluctuations of multiple pieces of generative information 410. For example, the storage processing function 155 may determine storage target data based on a rule, such as “if the fluctuation is equal to or greater than a predetermined standard, the prompt 50 is set as storage target data, and if the fluctuation is smaller than the predetermined standard, the prompt 50 and the generative information 410 are set as storage target data” or “if the fluctuation is smaller than a predetermined standard, the prompt 50 is set as storage target data, and if the fluctuation is equal to or greater than the predetermined standard, the prompt 50 and the generative information 410 are set as storage target data”.

If the user edits the prompt 50 after the warning is displayed by the display control function 156, the storage processing function 155 stores the edited prompt 50 as storage target data in the memory 120. If the user edits the prompt 50 after the warning is displayed, the second acquisition function 154 may input the edited prompt 50 to the generative model 40 to verify the reproducibility of the generative information 410. In this case, if the user performs an operation to specify the edited prompt 50 as a storage target after the display control function 156 displays, on the display 140, the generative information 410 generated by the generative model 40 with the edited prompt 50, the storage processing function 155 may store the edited prompt 50 as storage target data in the memory 120.

In this way, since the first information processing apparatus 100 in the present embodiment has the function to reduce fluctuations in the generative information 410, even when only the prompt 50 is stored and the generative information 410 is not stored, the generative information 410 can be reproduced with high accuracy from the stored prompt 50.

### Ninth Embodiment

In the eighth embodiment described above, the first information processing apparatus 100 inputs one prompt 50 to the same generative model 40 multiple times to obtain multiple pieces of generative information 410. However, the first information processing apparatus 100 may determine storage target data, based on the fluctuations in the generated multiple pieces of generative information 410.

The information processing system S in the present embodiment includes, for example, the first information processing apparatus 100, the second information processing apparatus 200, and the medical information system 300 in the same manner as in the first embodiment illustrated in FIG. 1. The first information processing apparatus 100 in the present embodiment includes the NW interface 110, the memory 120, the input interface 130, the display 140, and the processing circuitry 150, in the same manner as in the first embodiment. A configuration similar to that of the first embodiment is denoted by the same symbol and will not be further elaborated.

The processing circuitry 150 in the present embodiment includes the receiving function 151, the prompt generating function 152, the first acquisition function 153, the second acquisition function 154, the storage processing function 155, the display control function 156, and the deletion processing function 157, in the same manner as in the first embodiment. The processing circuitry 150 in the present embodiment may further include the determination function 159, in the same manner as in the fourth embodiment illustrated in FIG. 9.

For example, the determination function 159 may determine whether to store the prompt 50 and each of multiple pieces of generative information as storage target data in the memory 120, based on the similarity of the multiple pieces of generative information generated by inputting the same prompt 50 to the generative model 40 multiple times. In other words, the determination function 159 may determine data to be stored as storage target data in the memory 120.

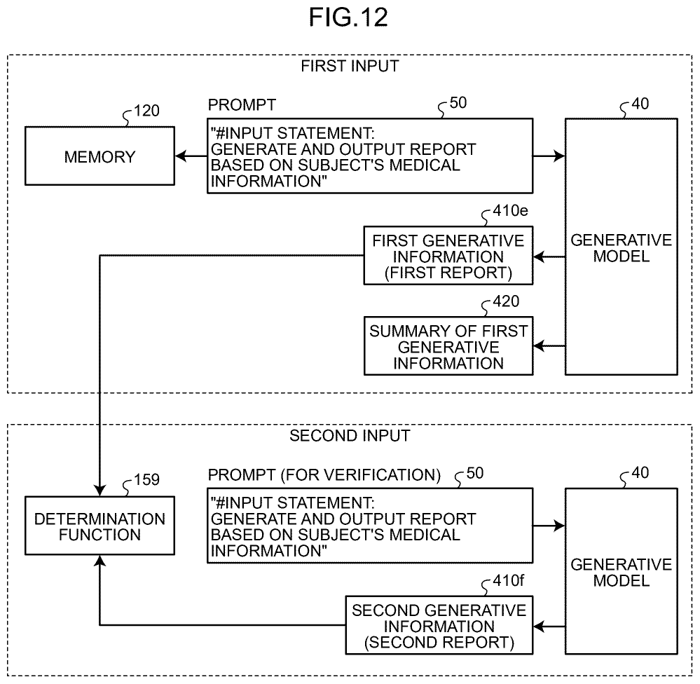

FIG. 12 is a diagram illustrating an example of determination of storage target data based on the similarity of multiple pieces of generative information according to the ninth embodiment. FIG. 12 represents a process overview, in which the second acquisition function 154 and the like are not illustrated.

In addition to a process similar to that of the first embodiment, the second acquisition function 154 in the present embodiment inputs the same prompt 50 to the same generative model 40 multiple times to acquire multiple pieces of generative information 410. For example, in FIG. 12, the second acquisition function 154 inputs the prompt 50 twice to the generative model 40.

In FIG. 12, the prompt 50 includes text “#input statement: generate and output a report based on the subject's medical information” as an example. First, in the first input, the second acquisition function 154 inputs the prompt 50 to the generative model 40. In this case, the generative model 40 outputs first generative information 410e. The generative model 40 may output a summary 420 of the first generative information 410e together with the first generative information 410e.

Then, in the second input, the second acquisition function 154 inputs the same prompt 50 as the first input to the generative model 40 for verification of the first generative information 410e. In this case, the generative model 40 outputs second generative information 410f based on the second input prompt 50.

In the example illustrated in FIG. 12, the first generative information 410e and the second generative information 410f are reports about the subject's medical information, because the prompt 50 includes an instruction to output a report. Thus, the first generative information 410e is also referred to as first report. The second generative information 410f is also referred to as second report. The first and second reports include text data describing, for example, the name of diagnosis for the subject and the site of a lesion. Depending on the content of the prompt 50, the first generative information 410e and the second generative information 410f may be, for example, image data indicating the site of a lesion.

When the description of the prompt 50 is ambiguous, inputting the same prompt 50 twice to the same generative model 40 may result in output data, for the first and second times, that are different from each other. When the generative model 40 has fluctuations, inputting the same prompt 50 may also result in output of different data. The fluctuations in the generative model 40 may occur when, for example, the amount of training data for the generative model 40 is insufficient or when there is a bias in the contents of training data for the generative model 40. Thus, the first generative information 410e and the second generative information 410f illustrated in FIG. 12 may be the same or different. When the first generative information 410e is different from the second generative information 410f, the difference between the first generative information 410e and the second generative information 410f may be minor or major.

The determination function 159 in the present embodiment therefore compares the first generative information 410e and the second generative information 410f to determine the similarity, and determines whether to store the prompt 50 as storage target data in the memory 120, based on the similarity.

For example, when the similarity between the first generative information 410e and the second generative information 410f is equal to or greater than a predetermined standard, it is likely that the prompt 50 is a suitable prompt that can provide stable and similar output data from the generative model 40. The determination function 159 in the present embodiment therefore determines to store the prompt 50 as storage target data in the memory 120 when the similarity between the first generative information 410e and the second generative information 410f is equal to or greater than a predetermined standard. In this case, the storage processing function 155 in the present embodiment stores the prompt 50 as storage target data in the memory 120, as illustrated in FIG. 12.

The method for determining the similarity between the first generative information 410e and the second generative information 410f by the determination function 159 is not particularly limited. For example, the determination function 159 may determine that the similarity is higher as the identicalness of the contents of medical reports is higher, such as that the names of diagnosis or the sites of lesions are common between the first generative information 410e and the second generative information 410f. Alternatively, the determination function 159 may determine the similarity between the first generative information 410e and the second generative information 410f by the percentage of words in common. When the first generative information 410e and the second generative information 410f are image data, the determination function 159 may determine the similarity based on the location, number, size, and the like of lesions in these pieces of image data.

The predetermined standard for the similarity between the first generative information 410e and the second generative information 410f, which is a criterion for determining whether to set the prompt 50 as storage target data, may be preset or may be specified by the user.

Between the first and second input processes, the prompt 50, the first generative information 410e, and the second generative information 410f may be saved temporarily in a volatile memory or in the memory 120, but not stored as storage target data in the memory 120. In other words, none of the prompt 50, the first generative information 410e, and the second generative information 410f is stored as storage target data in the memory 120 until storage target data is determined by the determination function 159. After storage target data is determined by the determination function 159, data other than the storage target data is deleted.

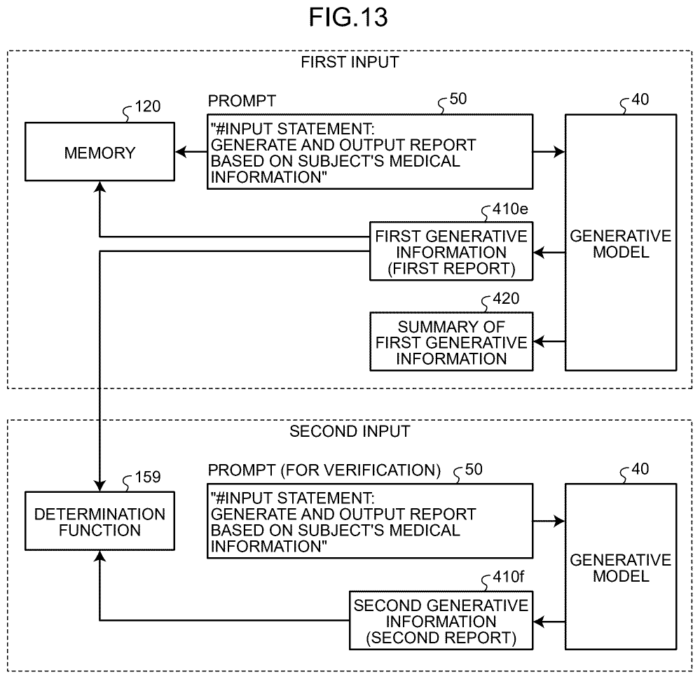

FIG. 13 is a diagram illustrating another example of determination of storage target data based on the similarity of multiple pieces of generative information according to the ninth embodiment.

The low similarity between the first generative information 410e and the second generative information 410f output from the generative model 40 with input of the same prompt 50 indicates that it is difficult to reproduce the first generative information 410e using the prompt 50. The determination function 159 in the present embodiment therefore determines not to store the prompt 50 as storage target data in the memory 120 when the similarity between the first generative information 410e and the second generative information 410f is less than a predetermined standard. In this case, the storage processing function 155 stores the first generative information 410e as storage target data in the memory 120. The storage processing function 155 may also store the summary of the first generative information 410e as storage target data in the memory 120. In this case, the storage processing function 155 does not store the prompt 50 in the memory 120.

If the data volume of the summary of the first generative information 410e is small, the summary of the first generative information 410e may be stored in the memory 120 even when the memory 120 stores the prompt 50 and does not store the first generative information 410e, as described with reference to FIG. 12.

The process of the determination function 159 in the present embodiment described with reference to FIGS. 12 and 13 may be divided and executed by multiple functions. For example, the evaluation function 158 may determine the similarity between the first generative information 410e and the second generative information 410f. The evaluation function 158 may evaluate the magnitude of fluctuations of multiple pieces of generative information 410 or the ambiguity of the prompt 50. The determination function 159 then may determine storage target data based on the evaluation result of the magnitude of fluctuations by the evaluation function 158.

In FIGS. 12 and 13, the determination function 159 determines storage target data. However, the user may make the final decision based on the determination result by the determination function 159. For example, the display control function 156 may display, on the display 140, the similarity between the first generative information 410e and the second generative information 410f as determined by the determination function 159. The display control function 156 may display the first generative information 410e and the second generative information 410f together with the similarity on the display 140. In this case, the receiving function 151 receives a user operation to select which of the prompt 50, the first generative information 410e, and the second generative information 410f is to be stored as storage target data in the memory 120. The storage processing function 155 stores data selected by the user among the prompt 50, the first generative information 410e, and the second generative information 410f in the memory 120.

The user may edit the prompt 50 in accordance with the similarity between the first generative information 410e and the second generative information 410f appearing on the display 140. For example, the display control function 156 displays the similarity between the first generative information 410e and the second generative information 410f and an edit screen for the prompt 50 on the display 140. In this case, the second acquisition function 154 inputs the prompt 50 edited by the user twice to the generative model 40 to obtain new first generative information 410e and new second generative information 410f based on the edited prompt 50. The determination function 159 then determines the similarity between the new first generative information 410e and the new second generative information 410f. The display control function 156 displays the newly determined similarity on the display 140. In this case, the editing of the prompt 50 and the process of determining the similarity between the first generative information 410e and the second generative information 410f may be repeated until the user decides to store the prompt 50 as storage target data in the memory 120.

The display control function 156 may display an example of the prompt with small fluctuations of output from the generative model 40, together with the edit screen for the prompt 50, on the display 140. An example of the prompt with small fluctuations of output from the generative model 40 may be, for example, a prompt 50 with a high similarity between the first generative information 410e and the second generative information 410f, among prompts 50 that were input to the generative model 40 in the past. The prompt 50 with a high similarity between the first generative information 410e and the second generative information 410f can be reworded as a prompt 50 that has a high likelihood of reproducing the first generative information 410e. The example of the prompt may be stored in the memory 120, for example. In this case, the display control function 156 reads the example of the prompt from the memory 120 and displays the read prompt on the display 140. In this case, the user can edit the prompt 50 by referring to the example of the prompt displayed.

The memory 120 may store multiple examples of the prompt. In this case, the display control function 156 may select one similar to the prompt 50 before editing by the user from among multiple examples of the prompt stored in the memory 120, and display the selected one on the display 140. For example, when the prompt 50 includes an instruction to generate and output a report, the display control function 156 may display, on the display 140, an example of the prompt that includes an instruction to generate and output a report from among multiple examples of the prompt. When the prompt 50 includes an instruction to generate and output image data, the display control function 156 may display, on the display 140, an example of the prompt that includes an instruction to generate and output image data from among multiple examples of the prompt.

In addition to manual editing of the prompt 50 by the user, automatic editing of the prompt 50 by the first information processing apparatus 100 may be possible. For example, the prompt generating function 152 of the processing circuitry 150 in the first information processing apparatus 100 may edit the prompt 50 such that fluctuations are reduced, based on an example of the prompt with smaller fluctuations of output from the generative model 40 that is stored in the memory 120.

For example, the prompt generating function 152 may automatically edit a prompt 50 by a trained model that has been trained by correlating an original prompt having large fluctuations with an edited prompt edited by the user so as to reduce the fluctuations of the original prompt. The second acquisition function 154 may input the automatically edited prompt 50 twice to the generative model 40 to obtain new first generative information 410e and new second generative information 410f. In this case, the determination function 159 determines the similarity between the new first generative information 410e and the new second generative information 410f. If the similarity is equal to or greater than a predetermined standard, the storage processing function 155 stores the automatically edited prompt 50 as storage target data in the memory 120. The automatic editing of the prompt 50 and the process of determining the similarity between the first generative information 410e and the second generative information 410f may be repeated until the similarity becomes equal to or greater than the predetermined standard.

### Tenth Embodiment

In the present tenth embodiment, existing medical information similar to the generative information 410 output from the generative model 40 is used as a reserve for a case where reproduction of the generative information 410 is inaccurate.

The information processing system S in the present embodiment includes, for example, the first information processing apparatus 100, the second information processing apparatus 200, and the medical information system 300 in the same manner as in the first embodiment illustrated in FIG. 1. The medical information system 300 in the present embodiment has a function as PACS, for example. The medical information system 300 therefore stores multiple pieces of medical image data.

The first information processing apparatus 100 in the present embodiment includes the NW interface 110, the memory 120, the input interface 130, the display 140, and the processing circuitry 150, in the same manner as in the first embodiment. A configuration similar to that of the first embodiment is denoted by the same symbol and will not be further elaborated.

The processing circuitry 150 in the present embodiment includes the receiving function 151, the prompt generating function 152, the first acquisition function 153, the second acquisition function 154, the storage processing function 155, the display control function 156, and the deletion processing function 157, in the same manner as in the first embodiment.

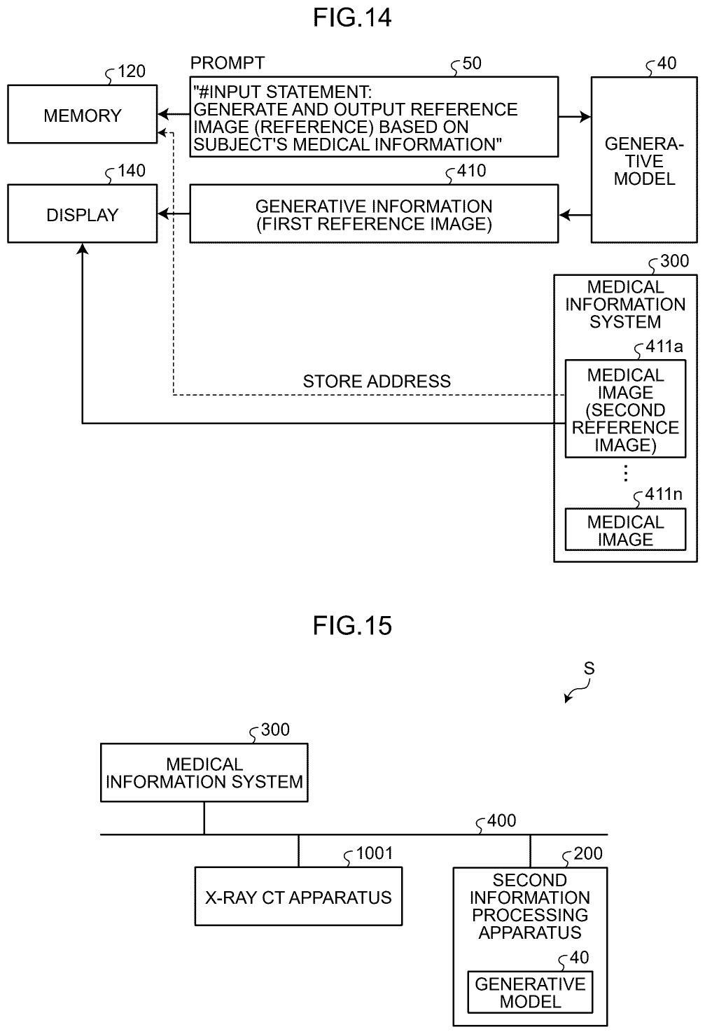

FIG. 14 is a diagram illustrating an example of storage target data according to the tenth embodiment.

The prompt 50 illustrated in FIG. 14 includes text “#input statement: generate and output a reference image (reference) based on the subject's medical information” as an example. In the present embodiment, the prompt 50 includes an instruction that causes a reference image to be output.

The second acquisition function 154 in the present embodiment inputs the prompt 50 to the generative model 40. In this case, the generative model 40 outputs a reference image as generative information 410, based on the prompt 50. In the present embodiment, the reference image output from the generative model 40 is also referred to as first reference image to be distinguished from a second reference image described later.

As described above, the medical information system 300 in the present embodiment stores multiple pieces of medical image data (hereinafter referred to as medical images 411a to 411n). In the present embodiment, among the multiple medical images 411a to 411n, one similar to the generative information 410 output from the generative model 40 is the second reference image serving as a reserve of the generative information 410. In the example illustrated in FIG. 14, the medical image 411a is the second reference image similar to the generative information 410.

As used herein, “the generative information 410 and the medical image 411a are similar” means that, for example, the organ to be imaged, the cross-sectional position in the subject, the cross-sectional direction, and the position, size, number, or the like of visualized abnormal parts (such as tumors) are identical or similar between the generative information 410 and the medical image 411a. The definition of similarity between the generative information 410 and the medical image 411a is not limited to this.

The multiple medical images 411a to 411n are, for example, images generated in advance based on medical images (medical-use images) of other subjects different from the “subject” in the prompt 50. In other words, the multiple medical images 411a to 411n are not the images dedicated to the prompt 50 for a certain subject, but are commonly referred to each time the generative model 40 is used. In other words, the medical images 411a to 411n are stored in the medical information system 300 as a database of reference medical images.

In addition to a process similar to that of the first embodiment, the first acquisition function 153 in the present embodiment acquires the medical image 411a similar to the generative information 410 from the medical information system 300 via the network 400 and the NW interface 110. For example, the first acquisition function 153 may identify one of the medical images 411a to 411n that is most similar to the generative information 410 by comparing the generative information 410 with the medical images 411a to 411n through known image processing.

The display control function 156 in the present embodiment displays the generative information 410 and the medical image 411a similar to the generative information 410 on the display 140.

The storage processing function 155 in the present embodiment stores the prompt 50 and the address indicating the save location where the medical image 411a is saved in the medical information system 300, as storage target data in the memory 120. The address indicating the save location where the medical image 411a is saved in the medical information system 300 is also referred to as save location information of the medical image 411a.

The storage processing function 155 stores the address indicating the save location where the medical image 411a similar to the generative information 410 is saved, as storage target data in the memory 120, but does not store the medical image 411a itself as storage target data in the memory 120. The storage processing function 155 does not store the generative information 410 as storage target data in the memory 120. The first information processing apparatus 100 in the present embodiment therefore can suppress increase in the amount of storage target data per input of the prompt 50 to the generative model 40.

For example, the user may re-input the prompt 50 stored as storage target data in the memory 120 to the generative model 40 at a later day in order to restore the generative information 410. In this case, the display control function 156 in the present embodiment displays, on the display 140, the medical image 411a indicated by the address stored as storage target data in the memory 120, together with the generative information 410 generated based on the re-input prompt 50. For example, due to fluctuations in the generative model 40, the generative information 410 generated based on the re-input prompt 50 may differ from the generative information 410 generated when the prompt 50 is first input. Even in this case, the display control function 156 displays, on the display 140, the medical image 411a similar to the generative information 410 generated when the prompt 50 is first input. Therefore, the user can check the medical image 411a similar to the initial generative information 410. In other words, the medical image 411a serves as a reserve in a case where the reproducibility of the generative information 410 is low due to fluctuations in the generative model 40.

In this way, the first information processing apparatus 100 in the present embodiment can store the address indicating the save location where the medical image 411a similar to the generative information 410 is saved as storage target data in the memory 120, thereby reducing increase in the amount of storage target data and ensuring security in a case where the reproducibility of the generative information 410 is low in the future.

### Eleventh Embodiment

In the first to tenth embodiments described above, the first information processing apparatus 100, which is a computer such as a server or a PC, is an example of the information processing apparatus. In the present eleventh embodiment, an X-ray CT apparatus executes a process as an example of the information processing apparatus.

FIG. 15 is a diagram illustrating an exemplary overall configuration of the information processing system S according to the eleventh embodiment. As illustrated in FIG. 15, the information processing system S in the present embodiment includes, as an example, an X-ray CT apparatus 1001, the second information processing apparatus 200, and the medical information system 300, which are communicatively connected via the network 400, such as a hospital LAN. The second information processing apparatus 200 and the medical information system 300 have functions similar to those of the first embodiment.

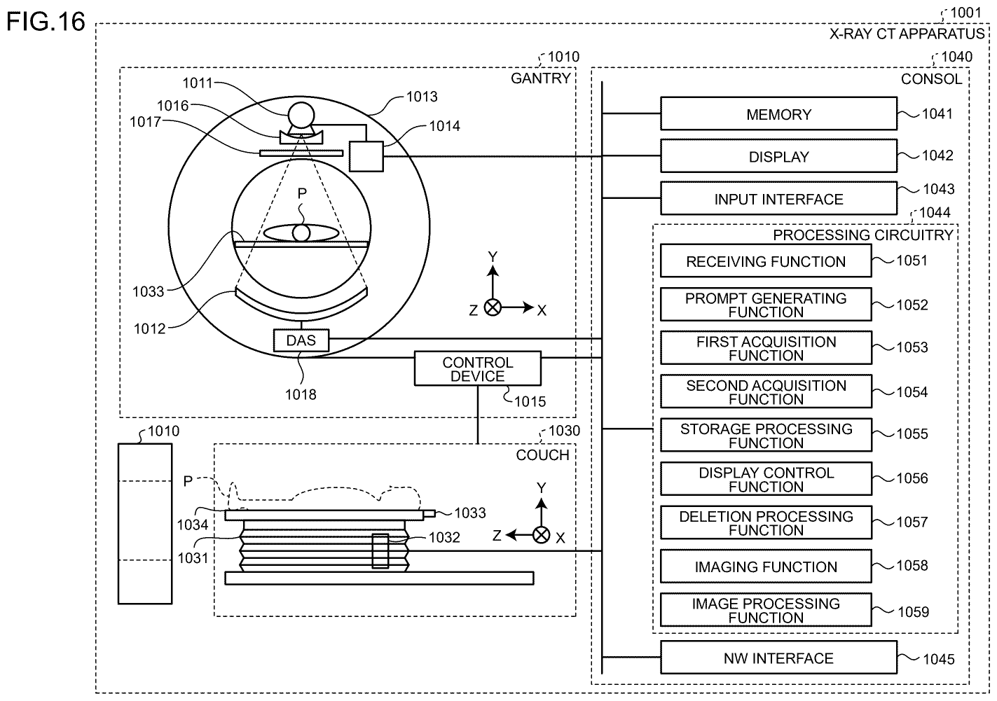

FIG. 16 is a diagram illustrating an exemplary hardware configuration of the X-ray CT apparatus 1001 according to the eleventh embodiment. The X-ray CT apparatus 1001 irradiates a subject (human subject) P with X-rays from an X-ray tube 1011 and detects the emitted X-rays with an X-ray detector 1012. The X-ray CT apparatus 1001 generates a CT image for the subject P based on an output from the X-ray detector 1012. There are various types of X-ray CT apparatus 1001, such as third-generation CT, fourth-generation CT, and fifth-generation CT. Any of these types can be applied to the present embodiment. Here, the third-generation CT is rotate/rotate type, in which the X-ray tube and the detector rotate as one unit around a subject. The fourth-generation CT is stationary/rotate type, in which a large number of X-ray detector elements arrayed in a ring shape are stationary and only the X-ray tube rotates around a subject. The fifth-generation system includes a focus coil that focuses an electron beam generated by an electron gun, a deflection coil that electromagnetically deflects the electron beam, and a target ring that surrounds half of the subject P and generates X-rays when the deflected electron beam collides.

As illustrated in FIG. 16, the X-ray CT apparatus 1001 includes a gantry 1010, a couch 1030, and a console 1040. In FIG. 16, the gantry 1010 is plurally depicted for convenience of illustration. The gantry 1010 is a scanning device having a configuration for X-ray CT imaging of the subject P. The couch 1030 is a transport device on which the subject P subjected to X-ray CT imaging is placed and which positions the subject P. The console 1040 is a computer that controls the gantry 1010. For example, the gantry 1010 and the couch 1030 are installed in a CT exam room, and the console 1040 is installed in a control room adjacent to the CT exam room. The gantry 1010, the couch 1030, and the console 1040 are communicatively connected to each other in a wired or wireless manner.

The console 1040 is not necessarily installed in the control room. For example, the console 1040 may be installed together with the gantry 1010 and the couch 1030 in the same room. The console 1040 may be incorporated into the gantry 1010.

In the present embodiment, the rotation axis of a rotary frame 1013 or the longitudinal direction of a couch top 1033 of the couch 1030 in a non-tilted state is defined as a Z-axis direction, the axis direction orthogonal to the Z-axis direction and horizontal to a floor surface is defined as an X-axis direction, and the axis direction orthogonal to the Z-axis direction and vertical to the floor surface is defined as a Y-axis direction.

As illustrated in FIG. 16, the gantry 1010 has the X-ray tube 1011, the X-ray detector 1012, the rotary frame 1013, an X-ray high voltage device 1014, a control device 1015, a wedge 1016, a collimator 1017, and a data acquisition system (DAS) 1018.

The X-ray tube 1011 is a vacuum tube having a cathode (filament) that generates thermal electrons and an anode (target) that generates X-rays upon collision of the thermal electrons. The X-ray tube 1011 emits thermal electrons from the cathode toward the anode using a high voltage supplied from the X-ray high voltage device 1014 to irradiate the subject P with X-rays.

Hardware for generating X-rays is not limited to the X-ray tube 1011. For example, instead of the X-ray tube 1011, the fifth-generation system may be used for generating X-rays.

The X-ray detector 1012 detects X-rays emitted from the X-ray tube 1011 and passing through the subject P, and outputs an electrical signal corresponding to the detected X-ray dose to the DAS 1018. The X-ray detector 1012 has, for example, an X-ray detector element array in which plural X-ray detector elements are arranged in the channel direction along one arc around the focus of the X-ray tube 1011. The X-ray detector 1012 has, for example, a structure in which plural X-ray detector elements in the channel direction are arranged in multiple rows in the slice direction (column direction and/or row direction). The X-ray detector 1012 is, for example, an indirect conversion type detector having a grid, a scintillator array, and an optical sensor array. The scintillator array includes a plurality of scintillators. The scintillators have a scintillator crystal that outputs light with a light quantity corresponding to an incident X-ray dose. The grid has an X-ray shielding plate disposed on a surface of the scintillator array on the X-ray incidence surface side and having a function of absorbing scattered X-rays. The grid may also be called collimator (one-dimensional collimator or two-dimensional collimator). The optical sensor array has a function of converting light from the scintillator into an electrical signal corresponding to the light quantity. For example, a photomultiplier tube (PMT) is used as the optical sensor. The X-ray detector 1012 may be a direct conversion type detector having a semiconductor device that converts incident X-rays into electrical signals. The X-ray detector 1012 is an example of a detection unit.

The rotary frame 1013 is an annular frame that supports the X-ray tube 1011 and the X-ray detector 1012 such that they face each other, and rotates the X-ray tube 1011 and the X-ray detector 1012 by the control device 1015 described later. An image field of view (FOV) is set at an opening of the rotary frame 1013. For example, the rotary frame 1013 is a casting made of aluminum. In addition to the X-ray tube 1011 and the X-ray detector 1012, the rotary frame 1013 can further support the X-ray high voltage device 1014, the wedge 1016, the collimator 1017, the DAS 1018, and the like. The rotary frame 1013 can further support various components not illustrated in FIG. 16.

The X-ray high voltage device 1014 has a high voltage generator and an X-ray controller. The high voltage generator has electrical circuitry including a transformer and a rectifier, and generates a high voltage to be applied to the X-ray tube 1011 and a filament current to be supplied to the X-ray tube 1011. The X-ray controller controls an output voltage in accordance with the X-rays emitted by the X-ray tube 1011. The high voltage generator may be a transformer system or an inverter system. The X-ray high voltage device 1014 may be provided in the rotary frame 1013 in the gantry 1010 or in a stationary frame (not illustrated) in the gantry 1010. The stationary frame is a frame that supports the rotary frame 1013 in a rotatable manner.

The control device 1015 includes a drive mechanism, such as a motor and an actuator, and processing circuitry having a processor for controlling the drive mechanism, a memory, and the like. The control device 1015 receives an input signal from an input interface 1043 or another input interface provided on the gantry 1010, and performs operation control for the gantry 1010 and the couch 1030. The operation control by the control device 1015 includes, for example, controlling the rotation of the rotary frame 1013, controlling the tilt of the gantry 1010, and controlling the operation of the couch 1030. The controlling the tilt of the gantry 1010 is implemented by the control device 1015 rotating the rotary frame 1013 around an axis parallel to the X-axis direction in accordance with inclination angle (tilt angle) information input by the input interface 1043 attached to the gantry 1010. The control device 1015 may be provided in the gantry 1010 or in the console 1040.

The wedge 1016 is a filter for regulating the dose of X-rays emitted from the X-ray tube 1011. Specifically, the wedge 1016 is a filter that transmits and attenuates the X-rays emitted from the X-ray tube 1011 such that the X-rays emitted from the X-ray tube 1011 toward the subject P have a predetermined distribution. For example, the wedge 1016 is a wedge filter or a bow-tie filter and is formed by processing aluminum or the like to have a predetermined target angle and a predetermined thickness.

The collimator 1017 restricts the irradiation range of the X-rays transmitted through the wedge 1016. The collimator 1017 slidably supports lead plates that block X-rays and adjusts the form of slits formed by the lead plates. The collimator 1017 may also be called an X-ray aperture.

The DAS 1018 reads, from the X-ray detector 1012, an electrical signal corresponding to the X-ray dose detected by the X-ray detector 1012. The DAS 1018 amplifies the read electrical signal and integrates (adds) the electrical signals over a view period to acquire detection data having a digital value corresponding to the X-ray dose over the view period. The detection data is called projection data. The DAS 1018 is implemented by, for example, an application specific integrated circuit (ASIC) with circuit elements capable of generating the projection data. The projection data is transmitted to the console 1040 via a non-contact data transmission device or the like. Here, the DAS 1018 is an example of a detection unit.

The detection data generated by the DAS 1018 is transmitted from a transmitter having a light emitting diode (LED) in the rotary frame 1013 to a receiver having a photodiode in a non-rotating part (e.g., stationary frame not illustrated in FIG. 16) of the gantry 1010 via optical communication, and then transferred to the console 1040. The method of transmitting the detection data from the rotary frame 1013 which is a rotating part to the non-rotating part of the gantry 1010 is not limited to the optical communication, and any method can be employed as long as it is non-contact data transfer.

In the present embodiment, the X-ray CT apparatus 1001 equipped with the integrated X-ray detector 1012 is described as an example. However, the technology according to the present embodiment can also be implemented as the X-ray CT apparatus 1001 equipped with a photon-counting X-ray detector.

The couch 1030 is a device for moving the subject P placed thereon to be scanned. The couch 1030 has a base 1031, a couch driver 1032, the couch top 1033, and a support frame 1034. The base 1031 is a housing that supports the support frame 1034 such that the support frame 1034 can be moved vertically. The couch driver 1032 is a drive mechanism that moves the couch top 1033 on which the subject P is placed in the longitudinal direction of the couch top 1033. The couch driver 1032 includes a motor and an actuator. The couch top 1033 is a plate on which the subject P is placed. The couch top 1033 is provided on a top surface of the support frame 1034. The couch top 1033 can protrude from the couch 1030 toward the gantry 1010 so that the whole body of the subject P can be imaged. The couch top 1033 is formed of, for example, a carbon fiber reinforced plastic (CFRP) that well transmits X-rays and has good physical properties such as rigidity and strength. For example, the interior of the couch top 1033 is hollow. The support frame 1034 supports the couch top 1033 such that the couch top 1033 can move in the longitudinal direction of the couch top 1033. In addition to the couch top 1033, the couch driver 1032 may move the support frame 1034 in the longitudinal direction of the couch top 1033.

The console 1040 has a memory 1041, a display 1042, an input interface 1043, processing circuitry 1044, and a NW interface 1045. Data communication between the memory 1041, the display 1042, the input interface 1043, the NW interface 1045, and the processing circuitry 1044 is performed via a bus. The console 1040 is described as a separate unit from the gantry 1010, but the gantry 1010 may include the console 1040 or part of components of the console 1040.

The memory 1041 is implemented by, for example, a semiconductor memory device such as ROM, RAM, or flash memory, a hard disk, or an optical disc. For example, the memory 1041 stores various storage target data. The memory 1041 also stores projection data and reconstructed image data. For example, the memory 1041 stores various computer programs. A storage area of the memory 1041 may be in the X-ray CT apparatus 1001 or in an external storage device connected via the network. Here, the memory 1041 is an example of a memory unit in the present embodiment.

The display 1042 is a liquid crystal display, a CRT display, or the like that displays various information.

The display 1042 may be provided anywhere in the control room. The display 1042 may be provided on the gantry 1010. The display 1042 may be a desktop type or may be configured as a tablet terminal or the like capable of wireless communication with a main body of the console 1040. One or two or more projectors may be used as the display 1042.

The input interface 1043 receives various input operations from the operator, converts the received input operations into electrical signals, and outputs the electrical signals to the processing circuitry 1044.

For example, a mouse, a keyboard, a trackball, a switch, a button, a joystick, a touchpad, and a touch panel display can be used as the input interface 1043. In the present embodiment, the input interface 1043 is not limited to those with these physical operating components. For example, an electrical signal processing circuit that receives an electrical signal corresponding to an input operation from an external input device installed separately from the apparatus and outputs the electrical signal to the processing circuitry 1044 is also an example of the input interface 1043. The input interface 1043 may be provided on the gantry 1010. The input interface 1043 may be configured as a tablet terminal or the like capable of wireless communication with the main body of the console 1040. Here, the input interface 1043 is an example of an input unit.

The NW interface 1045 is connected to the processing circuitry 1044 and controls transmission and communication of various data between the X-ray CT apparatus 1001, and the second information processing apparatus 200, and the medical information system 300. The NW interface 1045 is implemented by a network card, a network adapter, a NIC, or the like.

The processing circuitry 1044 controls the operation of the entire X-ray CT apparatus 1001. The processing circuitry 1044 has a processor and a memory such as ROM and RAM as hardware resources. The processing circuitry 1044 executes various system processes by the processor that executes a computer program loaded into the memory. The processing circuitry 1044 includes, for example, an receiving function 1051, a prompt generating function 1052, a first acquisition function 1053, a second acquisition function 1054, a storage processing function 1055, a display control function 1056, and a deletion processing function 1057. The receiving function 1051, the prompt generating function 1052, the first acquisition function 1053, the second acquisition function 1054, the storage processing function 1055, the display control function 1056, and the deletion processing function 1057 have functions similar to those of the receiving function 151, the prompt generating function 152, the first acquisition function 153, the second acquisition function 154, the storage processing function 155, the display control function 156, and the deletion processing function 157 in the processing circuitry 150 in the first embodiment.

The processing circuitry 1044 further includes an imaging function 1058 and an image processing function 1059. The imaging function 1058 and the image processing function 1059 are functions for capturing X-ray CT images.

For example, the imaging function 1058 controls a CT scan performed in the gantry 1010 to obtain X-ray detection data detected by the X-ray detector 1012. The imaging function 1058 performs reconstruction processing on the X-ray detection data to obtain X-ray CT image data in which the subject P is imaged. Examples of the reconstruction processing include filtered back projection, iterative reconstruction, and machine learning. The image processing function 1059 converts X-ray CT image data into any cross-sectional tomographic image data or any three-dimensional image data by a known method, based on an input operation received from the operator via the input interface 1043.

For example, the prompt generating function 1052 in the present embodiment may generate the prompt 50 including text “generate and output a similar case image based on subject's medical information” described with reference to FIG. 2 in the first embodiment, based on a user operation received by the receiving function 1051. The second acquisition function 1054 in the present embodiment inputs the prompt 50 generated by the prompt generating function 152 to the generative model 40 stored in the second information processing apparatus 200, and acquires the generative information 410 from the generative model 40.

The subject's medical information referred to by the prompt 50 in the present embodiment is, for example, CT image data captured by the X-ray CT apparatus 1001. In addition to the CT image data captured by the X-ray CT apparatus 1001, the prompt 50 may further refer to medical information such as patient's electronic medical record and test data stored in the medical information system 300.

Since the X-ray CT apparatus 1001 in the present embodiment has a function that allows the generative model 40 to generate the generative information 410 a plurality of different reference images can be generated from the CT image data generated in a single scan by the X-ray CT apparatus 1001. This configuration has the advantage of reducing imaging time and patient burden.

The subject's medical information referred to by the prompt 50 may be limited to the CT image data captured by the X-ray CT apparatus 1001. In this case, the processing circuitry 1044 in the present embodiment does not have to include the first acquisition function 1053 to acquire medical information from the medical information system 300. In this case, the information processing system S in the present embodiment does not have to include the medical information system 300. The generative model 40 may be installed in the X-ray CT apparatus 1001 instead of the second information processing apparatus 200. In this case, the X-ray CT apparatus 1001 can use the generative model 40 on a stand-alone basis.

In the present embodiment, the X-ray CT apparatus 1001 is an example of the information processing apparatus, but another medical image diagnostic apparatus may be an example of the information processing apparatus. For example, various medical image diagnostic apparatus, such as an X-ray diagnostic apparatus, a magnetic resonance imaging (MRI) apparatus, an ultrasound diagnostic apparatus, a single photon emission computed tomography (SPECT) apparatus, a positron emission computed tomography (PET) apparatus, a SPECT-CT apparatus that integrates the SPECT apparatus and the X-ray CT apparatus, and a PET-CT apparatus that integrates the PET apparatus and the X-ray CT apparatus, may have the functions of the processing circuitry 1044 illustrated in FIG. 16.

### First Modification

In the above embodiments, the information processing system S is installed in, for example, a medical institution such as a hospital but it may be installed in a company or the like other than a medical institution. In the above embodiments, an example in which the information processing system S is used in the medical field has been described. However, the field of application of the information processing system S is not limited to the medical field. The information processing system S can be used in various businesses and research and development that utilize generative AI. When used in a field other than the medical field, the information processing system S does not have to include the medical information system 300. For example, the information processing system S may include the first information processing apparatus 100 and the generative model 40. The information processing system S may include another database management apparatus or the like, instead of or in addition to the medical information system 300.

### Second Modification

In the above embodiments, as illustrated in FIG. 1 and the like, an example in which the first information processing apparatus 100, the second information processing apparatus 200, and the medical information system 300 are connected via the network 400 such as a hospital LAN has been described. However, the configuration of the information processing system S is not limited to this. For example, the second information processing apparatus 200 may be provided outside a medical institution. As a specific example, the second information processing apparatus 200 may be a server or the like of a business that provides generative AI as a service. In this case, the second information processing apparatus 200 may be connected to the first information processing apparatus 100 and the medical information system 300 via the Internet or the like instead of a hospital LAN.

### Third Modification

The generative model 40 may be provided in the first information processing apparatus 100 instead of the second information processing apparatus 200. For example, the generative model 40 may be stored in the memory 120 of the first information processing apparatus 100 and used by the second acquisition function 154 described above. Alternatively, the second acquisition function 154 may include the generative model 40.

### Fourth Modification

In the above embodiments, the generative model 40 is a multimodal model, but the technology for constructing the generative model 40 is not limited to this, and various AI models can be employed.

### Fifth Modification

In the above embodiments, as illustrated in FIG. 1 and the like, the memory 120 of the first information processing apparatus 100 stores storage target data. However, the storage target data may be saved outside the first information processing apparatus 100. For example, the information processing system S may include a dedicated information processing apparatus for storing the storage target data. In this case, a storage device outside the first information processing apparatus 100 is an example of a memory unit.

### Sixth Modification

In the first embodiment described above, only the generative information 410 is a target to be deleted based on a deletion condition, and the prompt 50 is not deleted. However, the prompt 50 may also be a target to be deleted based on a deletion condition. The deletion condition for the generative information 410 may be different from the deletion condition for the prompt 50.

### Seventh Modification

In the fourth embodiment described above, the determination function 159 determines whether to set the generative information 410 as storage target data based on the evaluation criterion for the clinical importance of the generative information 410. However, the determination criterion is not limited to this.

For example, the determination function 159 may determine the size of the data volume of the generative information 410 and determine whether to set the generative information 410 as storage target data in accordance with the data volume of the generative information 410. The data volume of the generative information 410 is an example of the property of the generative information 410 in the present modification. Specifically, the determination function 159 may determine that both the prompt 50 and the generative information 410 are storage target data if the data volume of the generative information 410 is less than a predetermined standard, and may determine that only the prompt 50 is storage target data when the data volume of the generative information 410 is equal to or greater than the predetermined standard. Instead of the determination function 159, the evaluation function 158 may evaluate the size of the data volume of the generative information 410.

When the prompt 50 includes image data or a large amount of medical information 21, its data volume may be greater than the generative information 410. The determination function 159 therefore may determine that only the generative information 410 is storage target data when the data volume of the prompt 50 is equal to or greater than the generative information 410, and may determine that only the prompt 50 is storage target data when the data volume of the prompt 50 is less than a predetermined standard.

The storage processing function 155 stores only the prompt 50, only the generative information 410, or both the generative information 410 and the prompt 50 as storage target data in the memory 120, based on the determination result by the determination function 159.

The embodiments and modifications described above may be combined. For example, any of the first to third and fifth to eleventh embodiments may be combined with the fourth embodiment. In this case, the storage processing function 155 may store the generative information 410 or the prompt 50 specified as storage target data by a user operation, and the generative information 410 or the prompt 50 determined as storage target data based on the evaluation result of the necessity to store, as storage target data in the memory 120.

The first and third embodiments may be combined. For example, the first information processing apparatus 100 may include the function of the storage processing function 155 in the first embodiment to automatically store the prompt 50 input to the generative model 40 as storage target data in the memory 120, and the function of the display control function 156 in the third embodiment to display an image button, for each piece of image data output from the generative model 40, that receives a user operation to specify the image data as a storage target on the display 140.

Various data handled herein are typically digital data.

According to at least one of the embodiments described above, the increase in the amount of saved data required for use of a generative model can be reduced.

While certain embodiments have been described, these embodiments have been presented by way of example only, and are not intended to limit the scope of the inventions. Indeed, the novel embodiments described herein may be embodied in a variety of other forms; moreover, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus, comprising:
an X-ray tube configured to irradiate a subject with X-rays;
an X-ray detector configured to detect X-rays emitted from the X-ray tube;
at least one memory; and
at least one piece of processing circuitry connected to the memory and configured to
generate a prompt, and
input the prompt to a generative model and acquire generative information from the generative model, wherein
the memory is configured to store, as storage target data, the prompt out of the generative information and the prompt corresponding to the generative information.

2. An information processing apparatus, comprising:
at least one memory; and
at least one piece of processing circuitry connected to the memory and configured to
generate a prompt, and
input the prompt to a generative model and acquire generative information from the generative model, wherein
the memory is configured to store, as storage target data, the prompt out of the generative information and the prompt corresponding to the generative information.

3. The information processing apparatus according to claim 2, wherein
the processing circuitry is configured to generate the prompt to include medical information, and
the generative information is a medical image.

4. The information processing apparatus according to claim 2, wherein the processing circuitry is configured to store the prompt as the storage target data in the memory when the prompt is input to the generative model.

5. The information processing apparatus according to claim 4, wherein the processing circuitry is configured to
receive a user operation to specify the generative information as the storage target data,
store the generative information as the storage target data in the memory in accordance with the user operation and store the prompt as the storage target data in the memory regardless of the user operation.

6. The information processing apparatus according to claim 2, wherein the processing circuitry is configured to
display icons on a display, the icons receiving a user operation to select, as the storage target data, one or both of the generative information and the prompt corresponding to the generative information, and
store, as the storage target data, one or both of the generative information and the prompt corresponding to the generative information in the memory in accordance with the user operation on the icons.

7. The information processing apparatus according to claim 6, wherein the icons include a prompt store button causing the prompt corresponding to the generative information to be stored as the storage target data in the memory.

8. The information processing apparatus according to claim 6, wherein the icons include a generative information store button causing the generative information to be stored as the storage target data in the memory.

9. The information processing apparatus according to claim 6, wherein the icons include a prompt display button that causing the prompt stored in the memory to be displayed on the display.

10. The information processing apparatus according to claim 2, wherein
the generative information includes image data,
the generative model outputs multiple pieces of the image data, and
the processing circuitry is configured to display icons on a display, the icons receiving, for each piece of the image data output from the generative model, a user operation to select the image data as the storage target data.

11. The information processing apparatus according to claim 2, wherein the processing circuitry is configured to
evaluate necessity to store the generative information,
determine whether to set the generative information as the storage target data based on an evaluation result, and
store, as the storage target data, the prompt or both the generative information and the prompt in the memory based on a determination result.

12. The information processing apparatus according to claim 11, wherein
the generative information includes information about a patient, and
the processing circuitry is configured to evaluate the necessity to store the generative information as being higher as a relevance between the generative information and a diagnostic result for the patient is stronger.

13. The information processing apparatus according to claim 2, wherein the processing circuitry is configured to
determine whether to set the generative information as the storage target data, in accordance with a data volume of the generative information, and
store, as the storage target data, the prompt or both the generative information and the prompt in the memory based on a determination result.

14. The information processing apparatus according to claim 2, wherein
the memory is a non-volatile storage device, and
the storage target data stored in the memory is not deleted except when a predetermined deletion condition is met or when a deletion operation is executed by a user.

15. The information processing apparatus according to claim 2, wherein the processing circuitry is configured to set the storage target data based on a magnitude of fluctuations of multiple pieces of the generative information generated by the generative model in response to input of the prompt.

16. The information processing apparatus according to claim 15, wherein the processing circuitry is configured to determine whether to set the generative information as the storage target data, based on an evaluation result of the magnitude of fluctuations.

17. The information processing apparatus according to claim 2, wherein the processing circuitry is configured to acquire restored generative information by inputting the prompt stored as the storage target data in the memory to the generative model, the restored generative information being restored based on the prompt by the generative model.

18. An information processing method comprising:
generating a prompt;
inputting the prompt to a generative model and acquiring generative information from the generative model; and
storing, as storage target data in a memory, the prompt out of the generative information and the prompt corresponding to the generative information.

19. A non-transitory computer readable recording medium on which programmed instructions are recorded, the instructions causing a computer to execute processing, the processing comprising:
generating a prompt;
inputting the prompt to a generative model and acquiring generative information from the generative model; and
storing, as storage target data in a memory, the prompt out of the generative information and the prompt corresponding to the generative information.
